SEQUENCE LISTING (26 sequences)

SEQ ID NO: 1 - DNA, 20 bp, Artificial Sequence
  Artificially synthesized
cgacacaccatgcaggccga

SEQ ID NO: 2 - DNA, 20 bp, Artificial Sequence
  Artificially synthesized
cctggtcaggccgccaatcg

SEQ ID NO: 3 - DNA, 21 bp, Artificial Sequence
  Artificially synthesized
tcaactccatcaagtcctctc

SEQ ID NO: 4 - DNA, 21 bp, Artificial Sequence
  Artificially synthesized
tcacattccttatcactgctg

SEQ ID NO: 5 - RNA, 19 nt, Artificial Sequence
  Artificially synthesized
gugaaauucuuggcuauua

SEQ ID NO: 6 - RNA, 19 nt, Artificial Sequence
  Artificially synthesized
gcgcuuggcugaaccauca

SEQ ID NO: 7 - RNA, 19 nt, Artificial Sequence
  Artificially synthesized
aagcugaccugauggaguu

SEQ ID NO: 8 - RNA, 19 nt, Artificial Sequence
  Artificially synthesized
cagcaaaucaugcgccuuu

SEQ ID NO: 9 - DNA, 25 bp, Artificial Sequence
  Artificially synthesized
caccgttggagagagagcgaggtat

SEQ ID NO: 10 - DNA, 25 bp, Artificial Sequence
  Artificially synthesized
aaacatacctcgctctctctccaac

SEQ ID NO: 11 - DNA, 25 bp, Artificial Sequence
  Artificially synthesized
caccgtgccacacaatggaactcgg

SEQ ID NO: 12 - DNA, 25 bp, Artificial Sequence
  Artificially synthesized
aaacccgagttccattgtgtggcac

SEQ ID NO: 13 - DNA, 25 bp, Artificial Sequence
  Artificially synthesized
caccgttggagagagaacgcggaat

SEQ ID NO: 14 - DNA, 25 bp, Artificial Sequence
  Artificially synthesized
aaacattccgcgttctctctccaac

SEQ ID NO: 15 - DNA, 25 bp, Artificial Sequence
  Artificially synthesized
caccggtcttcctgagaggtatgaa

SEQ ID NO: 16 - DNA, 25 bp, Artificial Sequence
  Artificially synthesized
aaacttcatacctctcaggaagacc

SEQ ID NO: 17 - DNA, 25 bp, Artificial Sequence
  Artificially synthesized
caccggtttatcttcagaatcagcc

SEQ ID NO: 18 - DNA, 25 bp, Artificial Sequence
  Artificially synthesized
aaacggctgattctgaagataaacc

SEQ ID NO: 19 - DNA, 25 bp, Artificial Sequence
  Artificially synthesized
caccgtacatctgctaaacatgagt

SEQ ID NO: 20 - DNA, 25 bp, Artificial Sequence
  Artificially synthesized
aaacactcatgtttagcagatgtac

SEQ ID NO: 21 - protein, 16 aa, Artificial Sequence
  Artificially synthesized
KLNSIKSSLGGETPGC

SEQ ID NO: 22 - protein, 115 aa, Artificial Sequence
  Artificially synthesized
GAELVRPGASVKLSCKASGYSFTNYWMNWVKQRPGQGLEWIGMIHPSDSETRLNQKFKDKATLTVDKSSSTAYMQLSSPTSEDSAVYYCAREGRLGLRSYAMDYWGQGTSVTVSS

SEQ ID NO: 23 - protein, 104 aa, Artificial Sequence
  Artificially synthesized
PSSLAMSVGQKVTMSCKSSQSLLNSSNQKNYLAWYQQKPGQSPKLLVYFASTRESGVPDRFVGSGSGTDFTLTITSVQAEDLADYFCQQHYITPLTFGAGTKLE

SEQ ID NO: 24 - protein, 14 aa, Artificial Sequence
  Artificially synthesized
CKVWKDATYSSKAR

SEQ ID NO: 25 - protein, 109 aa, Artificial Sequence
  Artificially synthesized
LQQSGPELVKPGASVKISCKASGYSFTGYFVNWVKQSHGKSLDWIGRIIPYNGDTFYNQKFKGKATLTVDKSSTTAHMELLSLTSEDSAVYYCGRGDYWGQGTSVTVSS

SEQ ID NO: 26 - protein, 105 aa, Artificial Sequence
  Artificially synthesized
PLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

(19) United States
(12) Patent Application Publication
(10) Pub. No.: US 2020/0179510 A1
(43) Pub. Date: Jun. 11, 2020

(54) LOW DENSITY LIPOPROTEIN RECEPTOR RELATED PROTEIN 5 INHIBITION SUPPRESSES TUMOR FORMATION

(71) Applicant: YALE UNIVERSITY, New Haven, CT (US)

(72) Inventors: Dianqing WU, Cheshire, CT (US); Qian XIAO, New Haven, CT (US)

(21) Appl. No.: 16/496,252

(22) PCT Filed: Feb. 16, 2018
(86) PCT No.: PCT/US18/00055

§ 371 Date: Sep. 20, 2019

Related U.S. Application Data
(60) Provisional application No. 62/476,109, filed on Mar. 24, 2017.

(51) Int. Cl. A61K 39/395; C07K 16/18; C07K 16/28; A61P 35/00

(52) U.S. Cl. CPC A61K 39/3955; C07K 16/18; A61K 2039/507; A61P 35/00; C07K 16/28

(57) ABSTRACT

The present invention relates to the discovery that inhibition of the interaction between Dickkopf2 (DKK2) and Low-Density Lipoprotein (LDL) Receptor Related Protein 5 (LRP5) and/or inhibition of LRP5 suppresses tumor formation. Thus, in various embodiments described herein, the methods of the invention relate to methods of treating cancer by administering to a patient an effective amount of an inhibiting agent that blocks the interaction between DKK2 and LRP5, methods of treating cancer by administering to a patient an effective amount of a LRP5 depleting agent, methods for providing anti-tumor immunity in a subject, and methods of stimulating a NK and T cell mediated immune response to a cell population or a tissue in a subject. Furthermore, the invention encompasses a pharmaceutical composition for treating cancer.

### CROSS-REFERENCE TO RELATED APPLICATIONS

The present application claims priority under 35 U.S.C. § 119(e) to U.S. Provisional Patent Application No. 62/476,109, filed Mar. 24, 2017, which application is hereby incorporated by reference in its entirety herein.

### STATEMENT REGARDING FEDERALLY SPONSORED RESEARCH OR DEVELOPMENT

This invention was made with government support under grants GM112182 and CA214703 awarded by the National Institutes of Health. The government has certain rights in the invention.

### BACKGROUND OF THE INVENTION

Cancer is a leading cause of death in humans. In the past decades remarkable advancements in cancer treatment and diagnosis have occurred. Treatment options for cancer include surgery, chemotherapy, radiation therapy, and immunotherapy. Most recently immunotherapy treatment, aimed at stimulating the immune system, has particularly attracted a lot of investigations. The immune system recognizes and is capable of suppressing cancer formation, but it is often held back by immune checkpoint pathways that are altered or misled by cancer to evade immune destruction. Immunotherapeutics that disrupt immune checkpoints, including anti-PD1, anti-CTLA4, and others in development, have shown clinical efficacy. Checkpoint inhibitors have been approved and shown evidence of efficacy in clinical trials for a number of tumors, including advanced melanoma, squamous NSCLC, and renal cell carcinoma, and Hodgkin's lymphoma, current checkpoint blockade inhibitors do not appear to be efficacious for colorectal cancer (CRC) (Brahmer, J., et al., 2012, N Engl J Med, 366: 2455-2465; Chung, K., et al., 2010, J Clin Oncol, 28: 3485-3490; Topalian, S. et al., 2015, Cancer Cell 27:450-461; Topalian, S. et al., 2012, N Engl J Med 366: 2443-2454). These variabilities in efficacy reflect differences in the known immune checkpoint mechanisms in different cancers and/or individual patients, and suggest the presence of yet to-be-discovered mechanisms of tumor evasion.

Although immunotherapy could be highly efficacious, only small subsets of patients regardless of the organ of origin of the tumor are usually responsive to therapy. New findings in this field are clearly needed for improving immunotherapy efficacy and specificity.

Wnt-signaling controls a wide variety of cell processes, including cell fate determination, differentiation, polarity, proliferation and migration. The Wnt family of secreted proteins bind to several classes of receptors, such as the low-density lipoprotein receptor related proteins 5 and 6 (LRP5/6), resulting in activation of several different intracellular signaling cascades, including the Wnt/β-catenin, Wnt/calcium and Wnt/Jnk pathways. Binding of Wnts to LRP5/6 specifically activates the Wnt/β-catenin pathway by blocking the function of a multiprotein complex that primes β-catenin for degradation, resulting in accumulation of 3-catenin in the cytoplasm and nucleus. Nuclear β-catenin complexes with members of the Lef/TCF family of transcription factors and activates gene expression. Although studies show that overexpression of LRP5 in non-physiological conditions can mediate Wnt-stimulated β-catenin signaling, in vivo loss of function studies suggest that LRP6 is the predominant Wnt co-receptor for regulating β-catenin signaling. Moreover, inactivation of the LRP5 gene has no detectable effect or marginal effects on Wnt-β-catenin signaling, depending on which tissue is analyzed.

Pathological states that may arise from altered stem cell function, such as degenerative diseases and cancer, are frequently associated with changes in Wnt/β-catenin pathway activity. Indeed, hyperactivation of the Wnt/β-catenin pathway is thought to induce premature senescence of stem cells and age-related loss of stem cell function (Brack et al., Science, 2007, Vol. 317 no. 5839 pp. 807-810; Liu et al., Science, 2007, Vol. 317 no. 5839 pp. 803-806). In cancer, hyperactivation of the Wnt/β-catenin pathway, often in conjunction with mutations in other cell growth regulatory genes, can lead to aberrant cell growth (Reya and Clevers, Nature, 2005, 434(7035):843-50). Thus, many ongoing investigations are focusing on Wnt/β-catenin pathway as a potential therapeutic target in cancer (Breuhahn et al., Oncogene, 2006, 25: 3787-3800; Greten et al., Br J Cancer, 2009, 100: 19-23). In particular, several research studies including cancer genomic sequencing projects revealed that more than 80% of colon cancers harbor a mutation or even a loss of the adenomatous polyposis coli (APC) gene, a major suppressor of the Wnt/β-catenin pathway (Kinzler and Vogelstein, Cell. 1996, Oct. 18; 87(2):159-70. Review; Sjoblom et al., Science, 2006, Oct. 13; 314(5797):268-74; Mann et al., Proc Natl Acad Sci USA, 1999. 96(4): p. 1603-8). APC and proteins such as GSK30 and Axin form a complex which marks β-catenin for degradation. Mutations in APC disrupt this complex and leads to increased levels of cytoplasmic β-catenin and its nuclear translocation. Since β-catenin is the most important adaptor of the Wnt signaling it promotes expression of oncogenic factors in response to Wnt ligands.

Wnt signaling is also regulated by a number of secreted polypeptide antagonists. These include four secreted Dickkopf (DKK) proteins (Monaghan et al., Mech Dev, 1999. 87: 45-56; Krupnik et al., Gene, 1999. 238: 301-13). Among these four DKK proteins, DKK1, 2 and 4 have been demonstrated to be effective antagonists of canonical Wnt signaling (Mao et al., Nature, 2001. 411: 321-5; Semenov et al., Curr Biol, 2001. 11: 951-61; Bafico et al., Nat Cell Biol, 2001. 3: 683-6; Niehrs, Nature, 2006. 25: 7469-81) by directly binding to Wnt coreceptor LRP 5/6 with high affinities (Mao et al., Nature, 2001. 411: 321-5; Semenov et al., Curr Biol, 2001. 11: 951-61; Bafico et al., Nat Cell Biol, 2001. 3: 683-6). Given that DKK proteins are Wnt antagonists, the conventional wisdom is that inactivation of DKK would increase Wnt activity and hence accelerate cancer formation.

The DKK molecules contain two conserved cysteine-rich domains (Niehrs, Nature, 2006. 25: 7469-81). Previously, it was shown that the second Cys-rich domains of DKK1 and DKK2 play a more important role in the inhibition of canonical Wnt signaling (Li et al., J Biol Chem, 2002. 277: 5977-81; Brott and Sokol Mol. Cell. Biol., 2002. 22: 6100-10). More recently, the structure of the second Cys-rich domain of DKK2 was solved and delineated amino acid residues on the domain that are required for DKK interaction with LRP5/6 and those for Kremens (Chen et al., J Biol Chem, 2008. 283: 23364-70; Wang et al., J Biol Chem, 2008. 283: 23371-5). DKK interaction with LRP5/6 underlie the primary mechanism for DKK-mediated inhibition of Wnt. Although DKK interaction with Kremen, also a transmembrane protein, was shown to facilitate DKK antagonism of Wnt signaling, this interaction may have other unresolved functions.

Wnt signaling is also mediated by the Wnt-co-receptor LRP 5/6. LRP5 plays a fundamental role in regulating bone mass. Loss of function LRP5 mutations have been shown to result in an autosomal recessive disorder characterized by low bone mass, whereas gain of function LRP5 mutations have been identified in autosomal dominant high bone mass. DKK proteins are associated with the regulation of bone formation and bone loss (in cancer and other diseases) via Wnt signaling. However, the possibility of DKK-mediated signaling via the Wnt co-receptor LRP5/6, without modifying Wnt signaling activity also has not been directly investigated.

Clearly there is a need of new ways to diminish cancer cell proliferation, to trigger cancer cell death, and to treat cancer. The current invention fulfills this need. Furthermore, the present invention satisfies the need for improving anti-cancer immunotherapy and cancer diagnosis.

### SUMMARY OF THE INVENTION

The present invention relates to compositions and methods of treating a cancer in a subject in need thereof. The method of treating a cancer comprises administering to the subject an effective amount of an inhibiting agent that blocks the interaction between Dickkopf 2 (DKK2) and Low-Density Lipoprotein (LDL) Receptor Related Protein 5 (LRP5), in a pharmaceutical acceptable carrier.

In another aspect, the invention includes a method for providing anti-tumor immunity in a subject. The method comprises administering to the subject an effective amount of an inhibiting agent that blocks the interaction between DKK2 and LRP5, with a pharmaceutical acceptable carrier. In another aspect, the invention provides a method for stimulating a T cell-mediated immune response to a cell population or tissue in a subject. The method comprises administering to the subject an effective amount of an inhibiting agent that blocks the interaction between DKK2 and LRP5, with a pharmaceutical acceptable carrier. In yet another aspect, the invention provides a method for stimulating a Natural Killer (NK) cell immune response to a cell population or tissue in a subject. The method comprises administering to the subject an effective amount of an inhibiting agent that blocks the interaction between DKK2 and LRP5, with a pharmaceutical acceptable carrier.

In some embodiments, the inhibiting agent is at least one selected from the group consisting of a DKK2 antagonist or fragment thereof, a DKK2 antibody or fragment thereof, a LRP5 antagonist or fragment thereof, a LRP5 antibody or fragment thereof, a siRNA, a ribozyme, an antisense molecule, an aptamer, a peptidomimetic, a small molecule, a CRISPR/Cas9 editing system and a combination thereof. In other embodiments the DKK2 antibody is 5F8.

In a further aspect, the invention includes a method of treating a cancer by administering to the subject an effective amount of a LRP5 gene depleting agent in a pharmaceutical acceptable carrier.

In another aspect, the invention includes a pharmaceutical composition for treating a cancer in a subject. The pharmaceutical composition of the present invention comprises a LRP5 depleting agent and a pharmaceutical acceptable carrier.

In yet another aspect, the invention provides a method for providing anti-tumor immunity in a subject. The method comprises administering to the subject an effective amount of a LRP5 antibody or fragment thereof with a pharmaceutical acceptable carrier. In a further aspect, the invention provides a method for stimulating a T cell-mediated immune response to a cell population or tissue in a subject. The method comprises administering to the subject an effective amount of a LRP5 antibody or fragment thereof with a pharmaceutical acceptable carrier. In some embodiments, the T cell-mediated immune response is a CD8⁺ cytotoxic T lymphocyte (CTL) response. In a further aspect, the invention provides a method for stimulating a Natural Killer (NK) cell immune response to a cell population or tissue in a subject. The method comprises administering to the subject an effective amount of a LRP5 antibody or fragment thereof with a pharmaceutical acceptable carrier.

In some embodiments, the LRP5 depleting agent is selected from the group consisting of a LRP5 antibody, a siRNA, a ribozyme, an antisense molecule, an aptamer, a peptidomimetic, a small molecule, a CRISPR/Cas9 editing system and a combination thereof. In other embodiments, the LRP5 depleting agent possesses neutralizing activity. In yet other embodiments, the LRP5 depleting agent does not affect canonical Wnt/β-catenin signaling.

In some embodiments, the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

In other embodiments, the cancer is selected from the group consisting of colorectal cancer, pancreatic cancer, gastric cancer, intestinal cancer, pancreatic cancer, esophageal cancer, skin cancer and lung cancer.

In some embodiments, the methods and composition of the invention comprise an additional agent selected from the group consisting of a chemotherapeutic agent, an anti-cell proliferation agent, an immunotherapeutic agent and any combination thereof. In further embodiments, the additional agent is a programmed cell death 1 (PD-1) antibody. In further embodiments, the LRP5 depleting agent and the additional agent are co-administered to the subject. In yet further embodiments, the route of administration is selected from the group consisting of inhalation, oral, rectal, vaginal, parenteral, topical, transdermal, pulmonary, intranasal, buccal, ophthalmic, intrathecal, and any combination thereof.

In some embodiments, the subject is a mammal. In other embodiments, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1G are series of histograms and images illustrating that DKK2 blockade decreases tumor burden in APCKO (APC^{min}DKK2^{-/-}) mice. FIG. 1 A-C. Genetic disruption of the DKK2 gene reduces tumor burdens in the APC^{Min/+} mice. Littermates were housed under specific pathogen-free condition for 20 weeks (female) or 22 weeks (male). FIG. 1A: Tumor/polyp number n>5,**P<0.01. FIG. 1B: Tumor/polyp size: APCKO tumors tend to be smaller than those of APC mice. n>5,**P<0.01; *P<0.05. FIG. 1C: Representative H and E staining reveals smaller and less frequent tumors in APCKO mice. FIG. 1D: ELISA showing 5F8 binds specifically to DKK2 protein, but not to DKK1. FIG. 1E: 5F8 antagonizes DKK2-mediated inhibition of Wnt reporter gene activity induced by Wnt3A. HEK293 cells were transfected with the Wnt reporter gene TOPFlash and treated with Wnt3A conditioned medium (CM), DKK2 CM or 5F8 (120 nM). FIG. 1F: 5F8 inhibits DKK2 binding to LRP5. HEK293 cells were transfected with LacZ (a control) or LRP5 expression plasmid. Binding of DKK2-AP fusion protein to cells in the presence or absence of 5F8 (120 nM) was measured. FIG. 1G. 5F8 reduces tumor burdens in APC^{Min/+} mice. Mice (10 weeks, female) were treated with 5F8 and IgG3 (8 mg/kg, twice a week, i.p.) for 8 weeks. Tumor/polyp number n=8,**P<0.01.

FIGS. 2A-2H are a series of graphs, histograms and images illustrating that DKK blockade suppresses tumor progression by increasing apoptosis, as measured by elevated granzyme B (gzmb) and Activated caspase 3 (Act. caspase 3), without altering cell proliferation or angiogenesis. FIGS. 2A-2B and FIGS. 2D-2G: A syngeneic mouse tumor model was used, in which C57BL mice were inoculated s.c. with 3×10³ MC38 cells. Treatment of 5F8 (10 mg/kg, every three days, i.p.) commenced at Day 14. FIG. 2A: Tumor volume and weight. Tumors were collected on Days, 14, 17, 20 and 22 for sizing. Tumors were collected on Day 22 for weighing. n=5, **P<0.01; n=5. FIG. 2B: Evaluation of survival. Mice treated with 5F8 had improved survival rates. n=10. FIG. 2C: 5F8 does not affect MC38 cell growth in culture. FIGS. 2D-2G. 5F8 treatment does not alter tumor angiogenesis (FIG. 2D) or tumor cell proliferation (FIG. 2E), but 5F8 treatment significantly increases apoptosis (FIG. 2F) and granzyme B-positive cells (FIG. 2G) within excised tumors. Sections of tumor collected in FIG. 2A were stained for CD31, Ki67, activated caspase 3, or granzyme B and counter-stained with DAPI. n=5, **P<0.01. FIG. 2H: DKK2-deficiency increases apoptosis and granzyme B-positive cells in the polyps of the APC^{Min/+} mice. Histological sections of polyps collected from the APC^{Min/+} and APC^{Min/+}DKK2^{-/-} mice (20 weeks) were stained with an activated caspase 3 antibody or a granzyme B antibody and DAPI. Scale bar is 150 μm.

FIGS. 3A-3J are a series of graphs and histograms demonstrating that DKK2 blockade enhances cytotoxic immune cell activation. FIGS. 3A-3B. 5F8 fails to alter tumor progression in NOD scid gamma (NSG) mice. MC38 cells (5×10³) were inoculated s.c in the NSG mice (n=5) and treatment with 5F8 or IgG control (10 mg/kg, every three days) commenced at Day 6. FIGS. 3C-3H. Flow cytometry analysis of tumor infiltrated leukocytes after an acute 5F8 treatment for 24 hours. MC38 cells (1×10⁵) were inoculated s.c in C57BL mice. When tumors reached an average size of 600 mm³, the mice were given one injection of 5F8 (10 mg/kg, i.p.). Tumors were collected in 24 hours for flow cytometry analysis. FIGS. 3C-3D: No change in cell population size of CD8+ cells or NK cells. FIGS. 3E-3F: Strong upregulation of granzyme b (gzmb) with 5F8 treatment over control IgG. FIGS. 3G-3H: Acute 5F8 treatment induced significant increases in activation markers for CD8+ and NK cells. FIG. 3C is pre-gated for CD45, whereas FIGS. 3D, 3E and 3G are derived from FIG. 3C. FIG. 3F is derived from FIG. 3D. MFI, mean fluorescence intensity. n=10 **P<0.01; *P<0.05. FIGS. 3I-3J: Depletion of NK1.1+ or CD8+ cells diminishes the tumor suppressive effect of 5F8. C57BL mice were inoculated s.c. with 5×10³ MC38 cells. For depletion of NK cells, anti-NK1.1 or isotype (Iso) control was injected i.p. at 300 ug/mouse at Day −1, 5, 11 and 17 of tumor cell inoculation. For CD8+ depletion, the anti-CD8a or isotype control was injected i.p. at 300 ug/mouse at Day 12, 15 and 19 of tumor cell inoculation. Treatment of 5F8 (10 mg/kg, every three days, i.p.) commenced at Day 12 for the NK cell depletion experiment or at Day 13 for CD8+ cell depletion experiment. n=5; **P<0.01; * P<0.05.

FIGS. 4A-4H are a series of graphs and histograms illustrating DKK2 suppression of NK cell activation. FIGS. 4A-4D: Treating co-culture of NK cells and tumor cells with 5F8 antibody increases granzyme b (gzmb) in NK cells and reduces tumor cell viability. Recapitulation of 5F8 effects on NK and tumor cells in their co-culture. Primary mouse NK cells expanded with IL-15 were added to YUMM1.7 or MC38 cells that were seeded one day before in the presence of 5F8 or IgG3 (250 nM) for 9 hours. Granzyme B expression in NK cells was examined by flow cytometry (FIG. 4A, FIG. 4D), whereas live tumor cells were determined by a Guava cell cytometer (FIGS. 4B-4C). FIG. 4A: Flow cytometry showing gzmb is upregulated in NK cells co-cultured with YUMM1.7 cells or with MC38 cells. FIGS. 4B-4C: Cytometry demonstrating a decrease in tumor cell viability when co-cultures are treated with 5F8, as compared with IgG control treatment. FIG. 4D: 5F8 treatment of primary NK cells alone did not enhance granzyme B production. FIGS. 4E-4F: DKK2 directly inhibits NK activation and production of granzyme B (gzmb). Isolated primary mouse NK cells were cultured with IL-15 (50 ng/ml) for 24 hrs. DKK2 protein (8 nM) were then added for another 24 hours, followed by flow cytometry analyses. n>3; **P<0.01; *P<0.05. FIG. 4G: DKK2-treated NK cells show reduced cytotoxic activity. Primary NK cells were expanded in IL-15 (50 ng/ml) for 24 hours followed by treatment with or without DKK2 (8 nM) for 24 hrs. The NK cells were then added to MC38 cells seeded the day before at 7:1 ratio. Numbers of apoptotic MC38 cells were determined after 6 hours, and numbers of live MC38 cells were determined after 9 hours of co-culture. “-” no addition. **P<0.01. FIG. 4H: WNT3A or GSK inhibitor does not affect NK activation. Isolated primary mouse NK cells were cultured with IL-15 (50 ng/ml) for 24 hrs. DKK2 protein (8 nM), WNT3 a (2 nM), and GSK3 inhibitor CHIR99021 (CHIR, 1 μM) were then added for another 24 hours, followed by flow cytometry analyses. n>3; **P<0.01; *P<0.05.

FIGS. 5A-5E are a series of images depicting DKK2 impedes phospho-STAT5 nuclear localization. FIGS. 5A-5C. DKK impairs phospho-STAT5 nuclear localization. Primary mouse NK cells were prepared and treated as in FIG. 4E. FIG. 5A: Western analysis shows reduced levels of granzyme b and perforin with DKK2 treatment. FIG. 5B: Cytosolic localization of phospho-STAT was detectable with DKK2 treatment. Immunostaining using anti-phospho-STAT5, anti-RAB8 (as a cytosol marker) and DAPI, followed by Alexa Fluor® 647 and FITC-labeled secondary antibodies. Scale bars are 5 μm. FIG. 5C: Cytosolic localization of phospho-STAT5 is reduced in NK cells isolated from 5F8-treated tumors. Tumor-infiltrated NK cells were isolated by FACS from MC38 tumors treated with IgG3 or 5F8 for 6 days (two 10 mg/kg injections). The cells were fixed, permeabilized and stained with anti-RAB8 (cytoplasm marker), anti-p-STAT5, and DAPI, followed by Alexa Fluor® 647 and FITC-labeled secondary antibodies. Scale bars are 5 μm. FIGS. 5D-5E: Phospho-STAT5 co-localizes with EEA1 in early/recycling endosomes, but not with late endosomal marker LAMP1, with DKK2 treatment. Primary mouse NK cells were prepared and treated as in A, followed by immunostaining using anti-phospho-STAT5, DAPI, and anti-EEA-1 (FIG. 5D) or anti-LAMP1 (FIG. 5E), followed by Alexa Fluor® 647 and FITC-labeled secondary antibodies. Scale bars are 5 μm.

FIGS. 6A-6I are a series of graphs, histograms and images demonstrating that LRP5 is required for DKK2-mediated inhibition of NK activation. FIGS. 6A-6B: Primary mouse NK cells were prepared from WT and LRP5−/− mice and treated as above, followed by flow cytometry and Western analysis (FIG. 6A) and immunostaining as described in FIG. 5B (FIG. 6B). FIG. 6A: DKK2 failed to inhibit NK cell activation in LRP5−/− cells. Western blot confirms that LRP5−/− cells lack LRP5 protein, but maintain normal levels of LRP6 protein expression. FIG. 6B: DKK2 failed to impair phospho-STAT5 localization in LRP5−/− cells, such that it localized to the nucleus rather than to endosomes. In WT cells, DKK2 treatment induced localization of phospo-STAT5 to endosomes. FIG. 6C: Hematopoietic LRP5-deficiency impairs grafted MC38 tumor progression and abrogates the effect of 5F8 on tumor progression. C57BL mice receiving LRP5f/fMX1Cre (LRP5−/−) or LRP5f/f (WT) bone marrows were treated with poly-I:C, followed by s.c. inoculation of 5×10³ MC38 cells. Treatment of 5F8 (10 mg/kg, i.p.) was given at Day 12, 17, and 20. n=5; **P<0.01. FIG. 6D: LRP5 intracellular domain C (LRP5C) and STAT5 co-immunoprecipitate in transfected HEK293 cells. FIGS. 6E-6F: LRP5C inhibits the STAT5 reporter gene activity that is induced by IL-15 in reconstituted HEK293 cells. The cells were infected with lentiviruses expressing JAK3, IL2/15Rβ, and the common γ subunit (Rγc). The cells were then transfected with the plasmid for LRP5 intracellular domain (LRP5C), the STAT5-luc reporter gene, and RFP (internal control) for 24 hrs. The cells were stimulated with IL-15 and IL15Rα-Fc for 6 hours before the reporter gene assay (FIG. 6E) and Western analysis (FIG. 6F). FIGS. 6G-6H: LRP5C inhibits the STAT5 reporter gene activity induced by activated JAK1 without affecting STAT5 phosphorylation. HEK293 cells were co-transfected with the STAT5 reporter gene plasmid and plasmids for activated JAK1 (JAK1CA, V658F) and LRP5C as indicated. After 24 hrs, the cells were analyzed for the reporter gene activity or by Western (FIG. 6G) or immunostaining with a phospho-STAT5 antibody and DAPI (FIG. 6H). Immunostained cells were examined by a confocal microscope and are presented with pseudocolor. Scale bars are 8 μm. FIG. 6I. DKK2 induces internalization of LRP5, but not LRP6. HEK293 cells were treated with DKK2 (4 nM) for times indicated. The cell surface proteins were biotinylated. Biotinylated cell surface proteins and cell lysate proteins were analyzed by Western blotting.

FIGS. 7A-7G are a series of graphs depicting the augmented anti-tumor effects and immune responses of DKK2 and PD-1 blockade combination. FIG. 7A: Augmented anti-tumor effects of DKK2 and PD-1 blockade combination in the MC38 tumor model. C57BL/6 mice were inoculated s.c. with MC38 cell. Treatment of 5F8 and/or anti-PD-1 (10 mg/kg, i.p) was done at every 5 days starting Day 18. Survival was evaluated by the Log-rank (Mantel-Cox) test (all significant differences are noted; *, <0.05; **, p<0.01). Individual tumor growth traces are shown in FIG. 13A. FIGS. 7B-7D: Effects of the antibody treatments on cytotoxic immune cells. 57BL/6 mice were inoculated s.c. with MC38 cell. Treatments with 5F8 and/or anti-PD-1 (10 mg/kg, i.p) were done at Days 13 and 18. Tumors were collected for flow cytometry analysis on Day 20. Data are presented as means±sem (*, <0.05; **, p<0.01; Anova test). FIG. 7E: Effect of recombinant DKK2 protein on cytotoxic immune cell responses to PD-1 blockade. C57BL/6 mice were inoculated s.c. with MC38 cell. When tumors grew to 500 mm3, they were injected with DKK2 protein (600 ng/25 μl/tumor; multiple injection sites per tumor) for three times every 8 hours. One hour after the last inject, tumors were collected, and infiltrated leukocytes were analyzed by flow cytometry. Data are presented as means±sem (*, <0.05; **, p<0.01; Anova test). FIG. 7F: Anti-tumor effects of DKK2 and PD-1 blockades in the YUMM1.7 tumor model. C57BL/6 mice were inoculated s.c. with YUMM1.7 cell. Treatment of 5F8 and/or anti-PD-1 (10 mg/kg, i.p) was done at every 5 days starting Day 12. Survival was evaluated by the Log-rank (Mantel-Cox) test (all significant differences are noted; *, <0.05; **, p<0.01). Mean and Individual tumor growth traces are shown in FIGS. 13D-13E. FIG. 7G: Effects of antibody treatments on cytotoxic immune cells. 57BL/6 mice were inoculated s.c. with YUMM1.7 cell. Treatments of 5F8 and/or anti-PD-1 (10 mg/kg, i.p) were done at Days 16 and 20. Tumors were collected for flow cytometry analysis on Day 21. Data are presented as means±sem (*, <0.05; **, p<0.01; Anova test).

FIGS. 8A-8G are a series of graphs and images illustrating the upregulation of DKK2 expression by APC-loss. FIG. 8A: Upregulation of DKK2 expression in human CRC samples over normal colorectal samples and in MSS CRCs over MSI CRCs. The numbers in the chart denote the sample sizes. FIGS. 8B-8C: Upregulation of DKK2 expression in mouse intestinal polyps. DKK2 mRNA levels were determined by quantitative RT-PCR using RNAs isolated from normal mouse intestines and polyps dissected from 24 weeks old APC^{Min/+} mice (B), whereas the DKK2 protein was detected by Immunostaining the intestinal sections using an anti-DKK2 antibody. FIGS. 8D-8E: Upregulation of DKK2 expression in APC-loss MC38 cells. DKK2 expression was determined by quantitative RT-PCR using RNAs isolated from MC38 cells with or without the APC mutation (FIG. 8D) or from APC mutant MC38 cells transfected with different β-catenin siRNAs (FIG. 8E). Western analysis of β-catenin levels is also shown. FIG. 8F: Upregulation of DKK2 expression in APC-loss HCT116 human colon cancer cells. DKK2 expression was determined by quantitative RT-PCR. FIG. 8G: Correlation between DKK2 expression and CRC patient survival rates. The overall and relapse-free survival rates of the high (top 15 percentile) and low (bottom 15 percentile) DKK2 expressers are compared using the TCGA provisional datasets of colorectal adenocarcinoma (FIG. 8A; n=56 for overall survival, n=50 for relapse-free survival) by the Mantel-Cox Log-Rank test.

FIGS. 9A-9R are a series of graphs, histograms and images depicting that 5F8 treatment activated granzyme b production in CD8+ and NK cells, without altering cell populations. FIGS. 9A-9G. Flow cytometry analysis of tumor infiltrated leukocytes. C57BL mice were inoculated s.c. with 5×10³ MC38 cells. Treatment of 5F8 (10 mg/kg, every three days, i.p.) commenced at Days 9 and 12. Tumors were collected at Day 14. They were digested by collagenase, and cells were analyzed by flow cytometry. FIGS. 9D-9F are derived from FIG. 9C, whereas FIG. 9G is derived from FIG. 9E. n=5; *P<0.05. FIG. 9A: 5F8 treatment suppresses tumor progression, as visualized by reduced tumor volume and weight, compared to control (IgG). FIGS. 9B-9E: No significant differences between 5F8 and its isotype-treated samples in the percentage of myeloid cells (Gr1^{high}CD11b^{high} or Gr1^{low}CD11b^{high}), CD4⁺, CD8⁺, T regulatory cells (CD4⁺ CD25⁺ Foxp3⁺), or NK1.1⁺ cells. FIGS. 9F-9G: 5F8 upregulated granzyme B in both CD8⁺ and NK1.1⁺ cells. FIGS. 9H-9K: Flow cytometry analysis of tumor draining lymph nodes. Inguinal lymph nodes were collected from mice described above and analyzed by flow cytometry. n=5. FIGS. 9H-9J: No significant differences in populations of CD4+, CD8+ or NK1.1+ cells resulted from treatment with 5F8.

FIG. 9I: Trend towards increased granzyme B in 5F8-treated CD8+ cells. FIG. 9K: Significant increase in granzyme B in 5F8-treated NK1.1.+ cells. FIGS. 9L-9O. Flow cytometry analysis of leukocytes in Peyer's patches (PPs) of APC^{Min/+} or APC^{Min/+} DKK2−/− mice (20 weeks old). FIGS. 9L-9M: Little difference between CD4+ or CD8+ cell populations in APC^{Min}/+ or APC^{Min/+} DKK2−/− mice, or from APC^{Min/+} mice injected with a dose of 5F8 (8 mg/kg, i.p.) for 24 hours (FIGS. 9N-90). FIGS. 9N-90: Strong increase in granzyme B-positive CD8+ cells in APC^{Min/+} mice treated with 5F8 over control. Populations shown are pre-gated for CD45.

FIGS. 9P-9R: Tumor-infiltrated leukocytes from FIGS. 3I-3J were analyzed by flow cytometry to confirm depletion efficiencies.

FIGS. 10A-10E are a series of graphs and a figure showing that DKK2 directly suppresses NK cell activation as related to FIGS. 4A-4H. FIG. 10A. DKK2 inhibits human NK cells. Human NK cells were isolated from peripheral bloods pooled from multiple normal individuals and incubated with human IL-15 (50 ng/ml) with or without 10 nM human DKK2 protein for 24 hour before analysis by flow cytometry. FIG. 10B. DKK2 inhibits IL-15-mediated activation of mouse primary CD8+ T cells. Primary CD8+ cells were isolated from spleens and cultured in IL-15+IL15Rα-Fc for 4 days. DKK2 (10 nM) was then added for 24 hours before flow analysis. Data are presented as means±sem (**, p<0.01; *<0.05; Student's t-test). FIG. 10C. DKK2 inhibits mouse IECs. Mouse IECs were isolated from normal mouse intestines and incubated with IL-15 (100 ng/ml) with or without 10 nM DKK2 protein for 24 hour before analysis by flow cytometry. FIG. 10D. TOPFLASH Wnt reporter gene assay. DKK2 (5 nM), and Wnt3a (2 nM), and GSK3 inhibitor CHIR (1 μM) were added for 6 hrs to cells transfected with TOPFLASH the day before. FIG. 10E. LRP5-deficiency does not affect WNT3A-induced accumulation of β-catenin in primary mouse NK cells. Isolated primary mouse NK cells were expanded with IL-15 (50 ng/ml) for 24 hrs and then incubated with WNT3A (5 nM) for 24 hours and analyzed by Western.

FIGS. 11A-11E are series of figures demonstrating that DKK2 suppresses NK cells via LRP5, but not LRP6, as related to FIGS. 6A-6I. FIG. 11A: LRP6 is not required for DKK2-mediated inhibition of NK activation. Primary mouse NK cells were prepared from WT and Lrp6−/− mice and treated as FIG. 6A, followed by flow cytometry and Western analysis. Data are presented as means±sem (**, p<0.01; *, p<0.05; Student's t-test). FIG. 11B: LRP6 is required for WNT3A-induced stabilization of b-catenin in NK cells. Mouse NK cells were treated as in FIG. 10E. FIGS. 11C-11D: Flow cytometry analyses of infiltrated leukocytes in tumors described in FIG. 6C. Data are presented as means±sem (**, p<0.01; *<0.05; n=5; Student's t-test). FIG. 11E: A model for DKK2 to provide tumor immune evasion. DKK2 produced by tumor cells and possibly tumor infiltrated stromal cells binds to LRP5 on NK cells, leading to sequestration of phospho-STAT5 to endosomes and reduction in its nuclear localization. This in turn leads to an impediment in NK cell activation including a reduction in granzyme B production and attenuated NK-mediated tumor cell killing.

FIGS. 12A-12C are a series of graphs illustrating corrections between DKK2 expression and patient survival rates, as related to FIG. 7. The overall and relapse-free survival rates of the high (top 15 percentile) and low (bottom 15 percentile) DKK2 expressers are compared using the TCGA provisional datasets of colorectal adenocarcinoma (FIG. 12A; n=56 for overall survival, n=50 for relapse-free survival), kidney renal papillary carcinoma (FIG. 12B; n=43 for overall survival, n=40 for relapse-free survival) and bladder urothelial carcinoma (FIG. 12C, n=61 for overall survival, n=48 for relapse-free survival) by the Mantel-Cox Log-Rank test.

FIGS. 13A-13D are series of graphs and images DKK2 impedes phospho-STAT5 nuclear localization related to FIGS. 5A-5E. FIGS. 13A-13B. Analysis of RNA sequencing results reveals relationship of DKK2-treatment to STAT signaling in mouse NK cells. Mouse NK cells were prepared and treated as FIG. 4D, and mRNAs isolated from these NK cells were subjected to sequencing. FIG. 13A shows pathway enrichment, whereas FIG. 13B shows alterations in STAT5 motif genes. Gene names are listed in FIG. 15. FIGS. 13C-13D: Individual channels of FIGS. 5B-5C.

FIGS. 14A-14G are a series of graphs illustrating the augmented anti-tumor effects of DKK2 and PD-1 combination blockade, as related to FIGS. 7A-7G. FIG. 14A: Individual tumor growth traces for FIG. 7A. FIG. 14B: Upregulation of DKK2 in human melanomas containing PTEN-loss and/or PI3K activated mutations. The numbers in the chart denote the sample size. FIG. 14C: A trend of upregulation of DKK2 expression in melanomas that are resistant to anti-PD-1 treatment. FIG. 14D: PI3K inhibitor Wortmannin reduces DKK2 expression in YUMM1.7 cells. The cells were treated with Wortmannin (5 μM) for 24 hours and DKK2 mRNA levels were determined by qRT-PCR. FIGS. 14E-14F. Individual and mean tumor growth traces for FIG. 7F. FIG. 14G: Additional results for FIG. 7G. FIG. 14H: Correlations between DKK2 expression and cancer patient survival rates. The survival rates of the high (top 15 percentile) and low (bottom 15 percentile) DKK2 expressers are compared using the TCGA provisional datasets of kidney renal papillary carcinoma (n=43) and bladder urothelial carcinoma (n=61) by the Mantel-Cox Log-Rank test.

FIG. 15 is a table listing the names of genes and statistics in the context RNA sequencing in mouse NK cells as detailed in FIGS. 13A-13C.

FIG. 16 is a list of nucleic acid sequences used herein as primers (SEQ ID NOs: 1-18).

FIG. 17 is a summary table listing key genes that suggest an alteration in STAT5 signaling after DKK2 treatment in the context of RNA sequencing in mouse NK cells (See FIGS. 13A-13C and FIG. 15).

### DETAILED DESCRIPTION OF THE INVENTION

The present invention relates to the unexpected discovery that inhibition of the interaction between Dickkopf 2 (DKK2) and Low-Density Lipoprotein (LDL) Receptor Related Protein 5 (LRP5), or direct inhibition of LRP5, results in suppression of tumor formation accompanied by increased cytotoxic activity of immune effector cells including natural killer (NK) cells and CD8⁺ cytotoxic T lymphocytes (CTLs) and increased tumor cell apoptosis. In various embodiments described herein, the methods of the invention relate to methods of treating cancer by administering to a patient an effective amount of (1) an inhibiting agent that blocks the interaction between DKK2 and LRP5 or (2) an LRP5 gene depleting agent, methods for providing anti-tumor immunity in a subject, methods of stimulating immune effector cell-mediated immune responses to a cell population or a tissue in a subject. Furthermore, the invention encompasses a pharmaceutical composition for treating cancer.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of +20% or +10%, more preferably +5%, even more preferably +1%, and still more preferably +0.1% from the specified value, as such variations are appropriate to perform the disclosed methods.

As used herein, “10% greater” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control.

As used herein, the terms “control,” or “reference” are used interchangeably, and refer to a value that is used as a standard of comparison (e.g., LRP5 level of expression in a healthy subject).

A “subject” or “patient,” as used therein, may be a human or non-human mammal. Non-human mammals include, for example, livestock and pets, such as ovine, bovine, porcine, canine, feline and murine mammals. Preferably, the subject is human.

A “mutation” as used therein is a change in a DNA sequence resulting in an alteration from its natural state. The mutation can comprise deletion and/or insertion and/or duplication and/or substitution of at least one deoxyribonucleic acid base such as a purine (adenine and/or thymine) and/or a pyrimidine (guanine and/or cytosine) Mutations may or may not produce discernible changes in the observable characteristics (phenotype) of an organism (subject).

The term “immunogenicity” as used herein, is the ability of a particular substance, such as an antigen or epitope, to provoke an immune response in the body of a mammal. This immune response could be humoral and/or cell-mediated.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process The term “activated T cells” indicates T cells that are currently undergoing cell division, cytokine production, performance of regulatory or cytolytic effector functions, and/or has recently undergone the process of “activation.” As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

The term the “immunotherapeutic agent” as used herein is meant to include any agent that modulates the patient's immune system. “immunotherapy” refers to the treatment that alters the patient's immune system.

The term “therapeutic” as used herein means a treatment and/or prophylaxis. A therapeutic effect is obtained by suppression, remission, or eradication of a disease state.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. This includes prevention of cancer.

The term “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

“DKK protein” refers to a protein of the DKK family of proteins that contains one or more cysteine-rich domains. The DKK family of proteins includes DKK1, DKK2, DKK3 and DKK4, and any other protein sufficiently related to one or more of these proteins at the sequence level, structurally or functionally. This family of proteins is described, e.g., in Krupnik et al. (1999) Gene 238:301. Allelic variants and mutants of DKK proteins such as those recited herein are also encompassed by this definition.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

“Granzyme B” refers to an enzyme from the granules of cytotoxic lymphocytes that, upon entry into the cytosol of a cell, induces apoptosis and/or nuclear DNA fragmentation.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes which contain certain mismatches, provided that the two strands are still forming a double stranded helix. “Stringent hybridization conditions” refers to hybridization conditions resulting in essentially specific hybridization. The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. An “isolated cell” or “isolated population of cells” is a cell or population of cells that is not present in its natural environment.

“LRP5” or “low density lipoprotein receptor-related protein 5” refers to all vertebrate nucleic acid and polypeptide forms of LRP5. LRP5 is a cell surface transmembrane receptor that functions in response to binding of ligands, such as DKK proteins. LRP5, along with co-receptor LRP6, may mediate canonical Wnt pathway signaling. LRP5 signaling may also occur independently of LRP6.

“LRP6” or “low density lipoprotein receptor-related protein 6” refers to all vertebrate nucleic acid and polypeptide forms of LRP6. LRP6 is a cell surface trans membrane receptor that functions in response to binding of ligands, such as DKK proteins. LRP6, along with co-receptor LRP5, may mediate canonical W t pathway signaling. LRP6 signaling may also occur independently of LRP5.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

“Perforin” refers to a protein that inserts into the membrane, generates oligomers and forms pores. Perforin permeabilizes the plasma membrane to allow entry of molecules, such as granzymes, into the target cell.

A “stem cell” refers to a cell that is capable of differentiating into a desired cell type. A stem cell includes embryonic stem (ES) cells; adult stem cells; and somatic stem cells, such as SP cells from uncommitted mesoderm. A “totipotent” stem cell is capable of differentiating into all tissue types, including cells of the meso-, endo-, and ecto-derm. A “multipotent” or “pluripotent” stem cell is a cell which is capable of differentiating into at least two of several fates.

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. The polypeptides generally will have significant amino acid identity relative to each other. A polymorphic variant is a variation in the polynucleotide sequence of a particular gene between individuals of a given species. Polymorphic variants may encompass “single nucleotide polymorphisms” (SNPs) in which the polynucleotide sequence varies by one base. The presence of SNPs may be indicative of, for example, a certain population, a disease state, or a propensity for a disease state.

The term “Wnt antagonist” or “Wnt inhibitor” refers to a molecule or composition which downregulates (e.g., suppresses or inhibits) signal transduction via the Wnt pathway. Downregulation may occur directly, e.g., by inhibiting a bioactivity of a protein in a Wnt signaling pathway, or indirectly, e.g., by inhibiting downstream mediators of Wnt signaling (such as TCF3) or by decreasing stability of β-catenin, etc. Examples of Wnt antagonists include, but are not limited to, DKK polypeptides (Glinka et al., Nature, 1998, 391: 357-62; Niehrs, Trends Genet, 1999, 15(8):314-9), crescent polypeptides (Marvin et al., Genes & Dev., 2001, 15: 316-327), cerberus polypeptides (U.S. Pat. No. 6,133,232), WISE/Sclerostin (Li et al., J Biol Chem, 2005. 280: 19883-7), axin polypeptides (Zeng et al., Cell, 1997, 90(1):181-92; Itoh et al., Curr Biol, 1998, 8(10):591-4; Willert et al., Development, 1999, 126(18):4165-73), Frzb polypeptides (Cadigan et al., Cell, 1998, 93(5):767-77; U.S. Pat. Nos. 6,133,232; 6,485,972), glycogen synthase kinase (GSK) polypeptides (He et al., Nature, 1995) 374(6523): 617-22), T-cell factor (TCF) polypeptides (Molenaar et al., Cell, 1996, 86(3):391-9), dominant negative dishevelled polypeptides (Wallingford et al., Nature, 2000, 405(6782): 81-5), dominant negative N-cadherin polypeptides (U.S. Pat. No. 6,485,972), dominant negative β-catenin polypeptides (U.S. Pat. No. 6,485,972), dominant negatives of downstream transcription factors (e.g., TCF, etc.), dominant negatives of Wnt polypeptides, agents that disrupt LRP-frizzled-wnt complexes, and agents that sequester Wnts (e.g., crescent and antibodies to Wnts). Wnt antagonist polypeptides may be of mammalian origin, e.g., human, mouse, rat, canine, feline, bovine, or ovine, or non-mammalian origin, e.g., from Xenopus, zebrafish, Drosophila, chicken, or quail. Wnt antagonists also encompass fragments, homologs, derivatives, allelic variants, and peptidomimetics of various polypeptides, including, but not limited to, DKK, crescent, cerberus, axin, Frzb, GSK, TCF, dominant negative dishevelled, dominant negative N-cadherin, and dominant negative β-catenin polypeptides. In other embodiments, Wnt antagonists also include antibodies (e.g., Wnt-specific antibodies), polynucleotides and small molecules.

The term “cancer” as used herein, includes any malignant tumor including, but not limited to, carcinoma, sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

The term “carcinoma” refers to a malignant new growth made up of epithelial cells tending to infiltrate surrounding tissues, and to give rise to metastases.

The term “cancer vaccine” refers to a vaccine that stimulates the immune system to fight a cancer or to fight the agents that contribute to the development of a cancer.

There are two broad types of cancer vaccines: Preventive cancer vaccines, which are intended to prevent cancer from developing in a healthy subject; and therapeutic cancer vaccines, which are intended to treat an existing cancer by strengthening the body's natural defenses against the cancer (Lollini et al., Nature Reviews Cancer, 2006; 6(3):204-216). As used herein the term “cancer vaccine” should be construed to include both preventive and therapeutic cancer vaccines.

The term “metastasis” refers to the spread of a cancer from one organ or part to another non-adjacent organ or part.

The term “angiogenesis” refers to the generation of new blood vessels, generally around or into a tissue or organ. Under normal physiological conditions, humans or animals undergo angiogenesis only in very specific restricted situations. For example, angiogenesis is normally observed in wound healing, fetal and embryonic development and formation of the corpus luteum, endometrium and placenta. Uncontrolled (persistent and/or unregulated) angiogenesis is related to various disease states, and occurs during tumor growth and metastasis.

The term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. For example, the skilled clinician will know that the size or rate of growth of a tumor can monitored using a diagnostic imaging method typically used for the particular tumor (e.g., using ultrasound or magnetic resonance image (MRI) to monitor a tumor).

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The term “antibody” or “Ab” as used herein, refers to a protein, or polypeptide sequence derived from an immunoglobulin molecule which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

By the term “synthetic antibody” as used herein, is meant an antibody generated using recombinant DNA technology, such as, for example, an antibody expressed by a bacteriophage as described herein. The term should also be construed to mean an antibody generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), VHH domains, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotide aptamer is a DNA or RNA molecule, usually comprising several strands of nucleic acids, that adopts highly specific three-dimensional conformation designed to have appropriate binding affinities and specificities towards specific target molecules, such as peptides, proteins, drugs, vitamins, among other organic and inorganic molecules. Such polynucleotide aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

The term “anti-tumor effect” as used herein, refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “xenograft” as used herein, refers to a graft of tissue taken from a donor of one species and grafted into a recipient of another species.

The term “allograft” as used herein, refers to a graft of tissue taken from a donor of one species and grafted into a recipient of the same species

“ShRNA” or “short hairpin RNA” as used herein refers to an interfering RNA sequence that is double stranded RNA capable of reducing or inhibiting the expression of a target gene or sequence when the ShRNA is in the same cell as the target gene or sequence. ShRNA may be produced continuously inside the target cell from a DNA construct, where the DNA construct may integrate into the target cell's nucleus or persist independently in the target cell. Accordingly, this DNA-directed ShRNA may continuously produce an interfering RNA within target cells.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The immune system is balanced between activation and suppression. Evasion of immunosurveillance is one of the prerequisites for tumor formation. One of the ways for tumors to evade immunosurveillance is to produce elevated levels of immunosuppressive molecules. Increasing number of immunosuppressive molecules and mechanisms have been identified over the years. Neutralization of these immunosuppressive molecules or their cognate signaling receptors has been shown to be efficacious in treating various malignancies.

The present invention relates to the discovery of a membrane bound receptor LRP5 that binds DKK proteins to suppresses natural killer (NK) cell and CD8⁺ cytotoxic T lymphocyte (CTL) activity, but does not affect canonical Wnt-β-catenin signaling in NK or CTL cells. Studies show that overexpression of LRP5, in non-physiological conditions, can mediate Wnt-induced stabilization of β-catenin and downstream β-catenin signaling; and, these LRP5-mediated effects can be inhibited by DKK. However, experimental evidence described herein demonstrates that LRP5, but not LRP6, has Wnt-independent signaling functions in NK and CTL cells. Experimental evidence disclosed herein indicates that LRP5 inhibitors and neutralizing antibodies are key immunomodulators and suppressors of tumor formation for treating cancers in which DKK is expressed. Thus LRP5 is a promising target for treating cancer.

### METHODS OF THE INVENTION

The present invention is directed to a method of treating cancer in a subject in need thereof. The method comprises administering to the subject an effective amount of an inhibiting agent that blocks the interaction between DKK2 and LRP5, in a pharmaceutical acceptable carrier. In some embodiments, the inhibiting agent is at least one selected from the group consisting of a DKK2 antagonist or fragment thereof, a DKK2 antibody or fragment thereof, a LRP5 antagonist or fragment thereof, a LRP5 antibody or fragment thereof, a siRNA, a ribozyme, an antisense molecule, an aptamer, a peptidomimetic, a small molecule, a CRISPR/Cas9 editing system and a combination thereof.

The present invention is also directed to a method of treating cancer in a subject in need thereof. The method comprises administering to the subject an effective amount of a LRP5 gene depleting agent in a pharmaceutical acceptable carrier. By the term “LRP5 gene depleting agent” is meant any agent that inhibits or reduces expression of LRP5 or that inhibits or reduces LRP5 activity in a cell, tissue or bodily fluid.

Small Interfering RNA (siRNA):

In one embodiment, the depleting agent is a small interfering RNA (siRNA). siRNA is an RNA molecule comprising a set of nucleotides that is targeted to a gene or polynucleotide of interest. As used herein, the term “siRNA” encompasses all forms of siRNA including, but not limited to (i) a double stranded RNA polynucleotide, (ii) a single stranded polynucleotide, and (iii) a polynucleotide of either (i) or (ii) wherein such a polynucleotide, has one, two, three, four or more nucleotide alterations or substitutions therein. siRNAs and their use for inhibiting gene expression are well known in the art (Elbashir et al., Nature, 2001, 411 (6836): 494-988). In the present invention the siRNA is capable of interfering with the expression and/or the activity the gene of interest such as LRP5.

### Ribozyme:

In a further embodiment, the depleting agent is a ribozyme. Ribozymes and their use for inhibiting gene expression are also well known in the art (Cech et al., 1992, J. Biol. Chem. 267:17479-17482; Hampel et al, 1989, Biochemistry 28:4929-4933; Eckstein et al., International Publication No. WO 92/07065; Altaian et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific. There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes useful for inhibiting the expression of a gene of interest (i.e. LRP5) may be designed by incorporating target sequences into the basic ribozyme structure which are complementary to the mRNA sequence of the desired gene. Ribozymes targeting the gene of interest may be synthesized using commercially available reagents (Applied Biosystems, Inc., Foster City, Calif.) or they may be genetically expressed from DNA encoding them.

### Antisense Molecule:

In another embodiment, the depleting agent is an antisense nucleic acid sequence. Antisense molecules and their use for inhibiting gene expression are well known in the art (Cohen, 1989, Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes. An antisense molecules may be provided to the cell via genetic expression using DNA encoding the antisense molecule as taught by Inoue, 1993, U.S. Pat. No. 5,190,931. Alternatively, antisense molecules may be made synthetically and then provided to the cell. Antisense oligomers of between about 10 to about 30, are preferred, since they are easily synthesized and introduced into a target cell. Synthetic antisense molecules contemplated by the invention include oligonucleotide derivatives known in the art which have improved biological activity compared to unmodified oligonucleotides (U.S. Pat. No. 5,023,243).

### CRISPR/Cas9 System

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in, for example, cell lines (such as 293T cells) or primary cells. The CRISPR1Cas9 system can simultaneously target multiple genomic loci by co-expressing a single Cas9 protein with two or more gRNAs, making this system uniquely suited for multiple gene editing or synergistic activation of target genes.

### Small-Molecule Inhibitors

It is well known in the art that some amino acid residues, located at the top cavity of the β-propeller structure of the third YWTD repeat domain of human LRP5, are important for DKK binding and DKK-mediated Wnt antagonism (Zhang et al., Mol Cell Biol. 2004; 24:4677-4684). In one embodiment of the present invention is a small molecule, which can disrupt the interaction between DKK2 and LRP5 and which acts as a LRP5 inhibiting agent that does not affect canonical Wnt-β-catenin signaling via Wnt co-receptor LRP 5/6.

### Antibodies

The invention contemplates using a composition comprising an anti-DKK2 antibody (e.g. 5F8, SEQ ID NOs: 21-23) and/or an anti-LRP5 antibody as an agent that blocks the interaction between DKK2 and LRP5. In one embodiment, the antibody comprises an antibody selected from a polyclonal antibody, a monoclonal antibody, a humanized antibody, a synthetic antibody, a heavy chain antibody, a human antibody, and a biologically active fragment of an antibody and any combination thereof.

Methods of producing antibodies are known in the art. Exemplary techniques for the production of the antibodies used in accordance with the present invention are herein described. It will be appreciated by one skilled in the art that an antibody comprises any immunoglobulin molecule, whether derived from natural sources or from recombinant sources, which is able to specifically bind to an epitope present on a target molecule. In one embodiment, the target molecule comprises

When the antibody to the target molecule used in the compositions and methods of the invention is a polyclonal antibody (IgG), the antibody is generated by inoculating a suitable animal with a peptide comprising full length target protein, or a fragment thereof, an upstream regulator, or fragments thereof. These polypeptides, or fragments thereof, may be obtained by any methods known in the art, including chemical synthesis and biological synthesis.

Antibodies produced in the inoculated animal that specifically bind to the target molecule, or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow et al., 1998, In: Antibodies, A Laboratory Manual, Cold Spring Harbor, N.Y.

Monoclonal antibodies directed against a full length target molecule, or fragments thereof, may be prepared using any well-known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. Patent Publication No. 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al., 1992, Critical Rev. Immunol. 12(3,4):125-168, and the references cited therein.

When the antibody used in the methods of the invention is a biologically active antibody fragment or a synthetic antibody corresponding to antibody to a full length target molecule, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology available in the art, and described, for example, in Wright et al., 1992, Critical Rev. in Immunol. 12(3,4): 125-168 and the references cited therein. Alternatively, quantities of the desired antibody or fragment thereof may also be synthesized using chemical synthesis technology. If the amino acid sequence of the antibody is known, the desired antibody can be chemically synthesized using methods known in the art.

The present invention also may include the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen et al. (U.S. Pat. No. 6,180,370), Wright et al., 1992, Critical Rev. Immunol. 12(3,4):125-168, and in the references cited therein, or in Gu et al., 1997, Thrombosis & Hematocyst 77(4):755-759, or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, 1979, which is incorporated herein by reference).

DNA sequences of human antibodies and particularly the complementarity determining regions (CDRs) can be isolated in accordance with procedures well known in the art. Preferably, the human CDRs DNA sequences are isolated from immortalized B-cells as described in International Patent Application Publication No. WO 1987/02671. CDRs useful in producing the antibodies of the present invention may be similarly derived from DNA encoding monoclonal antibodies capable of binding to the target molecule. Such humanized antibodies may be generated using well-known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

Another method of generating specific antibodies, or antibody fragments, reactive against a LRP5 involves the screening of expression libraries encoding immunoglobulin genes, or portions thereof, expressed in bacteria with a LRP5 protein or peptide. For example, complete Fab fragments, VH regions and Fv regions can be expressed in bacteria using phage expression libraries. See for example, Ward et al., Nature, 1989, 341: 544-546; Huse et al., Science, 1989, 246: 1275-1281; and McCafferty et al., Nature, 1990, 348: 552-554. Screening such libraries with, for example, a DKK2 or a LRP5 peptide, can identify immunoglobulin fragments reactive with DKK2 or LRP5. Alternatively, the SCID-hu mouse (available from Genpharm) can be used to produce antibodies or fragments thereof.

In a further embodiment, antibodies or antibody fragments can be isolated from antibody phage libraries generated using the techniques described in McCafferty et al., Nature, 1990, 348: 552-554. Clackson et al., Nature, 1991, 352: 624-628 and Marks et al., J Mol Biol, 1991, 222: 581-597 describe the isolation of murine and human antibodies, respectively, using phage libraries. Subsequent publications describe the production of high affinity (nM range) human antibodies by chain shuffling (Marks et al., BioTechnology, 1992, 10: 779-783), as well as combinatorial infection and in vivo recombination as a strategy for constructing very large phage libraries (Waterhouse et al., Nuc. Acids. Res., 1993, 21: 2265-2266). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 1984, 81: 6851), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen combining site of an antibody to create a chimeric bivalent antibody having one antigen-combining site with specificity for a first antigen and another antigen-combining site with specificity for a different antigen.

Various techniques have been developed for the production of functional antibody fragments. The antibody fragment may include a variable region or antigen-binding region of the antibody. Traditionally, these fragments were derived via proteolytic digestion of intact antibodies (see, e.g., Morimoto et al., Journal of Biochemical and Biophysical Methods, 1992, 24: 107-117 and Brennan et al., Science, 1985, 229: 81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′) 2 fragments (Carter et al., Bio/Technology, 1992, 10: 163-167). According to another approach, F (ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody”, e.g., as described in U.S. Pat. No. 5,641,870 for example. Such linear antibody fragments may be monospecific or bispecific.

Antibody mimics or “non-antibody binding protein” use non-immunoglobulin protein scaffolds, including adnectins, avimers, single chain polypeptide binding molecules, and antibody-like binding peptidomimetics by using non-immunoglobulin protein scaffolds as alternative protein frameworks for the variable regions of antibodies (U.S. Pat. Nos. 5,260,203; 5,770,380; 6,818,418 and 7,115,396). Other compounds have been developed that target and bind to targets in a manner similar to antibodies. Certain of these “antibody mimics” use non-immunoglobulin protein scaffolds as alternative protein frameworks for the variable regions of antibodies. A methodology for reducing antibodies into smaller peptidomimetics, termed “antibody like binding peptidomimetics” (ABiP) can be used, a methodology for reducing antibodies into smaller peptidomimetics, can also be useful as an alternative to antibodies (Murali et al. Cell Mol Biol., 2003, 49(2):209-216).

Fusion proteins that are single-chain polypeptides including multiple domains termed “avimers” were developed from human extracellular receptor domains by in vitro exon shuffling and phage display and are a class of binding proteins somewhat similar to antibodies in their affinities and specificities for various target molecules (Silverman et al. Nat Biotechnol, 2005, 23: 1556-1561). The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds including, but not limited to, RNA molecules and unnatural oligomers (e.g., protease inhibitors, benzodiazepines, purine derivatives and beta-turn mimics) all of which are suitable for use with the present invention. These are aimed to circumvent the limitations of developing antibodies in animals by developing wholly in vitro techniques for designing antibodies of tailored specificity.

As known in the art, aptamers are macromolecules composed of nucleic acid that bind tightly to a specific molecular target. Tuerk and Gold (Science, 1990, 249:505-510) discloses SELEX (Systematic Evolution of Ligands by Exponential Enrichment) method for selection of aptamers. In the SELEX method, a large library of nucleic acid molecules (e.g., 1015 different molecules) is produced and/or screened with the target molecule. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology.

The term “neutralizing” in reference to an anti-DKK2 and/or anti-LRP5 antibody of the invention or the phrase “antibody that neutralizes DKK2 activity” or “antibody that neutralizes LRP5 activity” is intended to refer to an antibody whose binding to or contact with LRP5 results in inhibition of a cell proliferative activity, metastasis of cancer, invasion of cancer cells or migration of cancer cells, establishment of tumor-formation promoting microenvironment induced by DKK2 and/or LRP5. Because the DKK2 is secreted extracellularly and functions as an essential factor of proliferation, migration, invasion and metastasis of cancer cells, some anti-DKK2 antibodies and/or LRP5 antibodies may neutralize these activity. The neutralizing antibody in this invention is especially useful in therapeutic applications: to prevent or treat intractable diseases cancers, and cancer metastasis. In some embodiments, the neutralizing antibody in this invention can be administered to a patient, or contacted with a cell for inhibiting metastasis of a cancer characterized by the over-expression of DKK2.

The antibody of the present invention can be assessed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York, 2002).

### Combination Therapies

The compounds identified in the methods described herein may also be useful in the methods of the invention when combined with at least one additional compound useful for treating cancer. The additional compound may comprise a compound identified herein or a compound, e.g., a commercially available compounds, known to treat, prevent, or reduce the symptoms of cancer and/or metastasis.

In one aspect, the present invention contemplates that the agents useful within the invention may be used in combination with a therapeutic agent such as an anti-tumor agent, including but not limited to a chemotherapeutic agent, immunotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine.

Other examples of chemotherapeutic agents include, but are not limited to, the following and their pharmaceutically acceptable salts, acids and derivatives: nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK@ razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and docetaxel (TAXOTERE, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

An immunotherapeutic agent may be, but is not limited to, an interleukin-2 or other cytokine, an inhibitor of programmed cell death protein 1 (PD-1) signaling such as a monoclonal antibody that binds to PD-1, Ipilimumab. The immunotherapeutic agent can also block cytotoxic T lymphocytes associated antigen A-4 (CTLA-4) signaling and it can also relate to cancer vaccines and dendritic cell-based therapies.

The immunotherapeutic agent can further be NK cells that are activated and expanded by means of cytokine treatment or by transferring exogenous cells by adoptive cell therapy and/or by hematopoietic stem cell transplantation. NK cells suitable for adoptive cell therapy can be derived from different sources, including ex vivo expansion of autologous NK cells, unstimulated or expanded allogeneic NK cells from peripheral blood, derived from CD34+ hematopoietic progenitors from peripheral blood and umbilical cord blood, and NK-cell lines. Genetically modified NK cells expressing chimeric antigen receptors or cytokines are also contemplated in this invention. Another immunotherapeutic agent useful for this invention is an agent based on adoptive T cell therapy (ACT) wherein tumor-infiltrating lymphocytes (TILs) are administered to patients. The administered T cells can be genetically engineered to express tumor-specific antigen receptors such as chimeric antigen receptors (CARs), which recognize cell-surface antigens in a non-major histocompatibility (MHC)-restricted manner; or they can be traditional αβ TCRs, which recognize epitopes of intracellular antigens presented by MHC molecules.

### Pharmaceutical Compositions and Formulations.

The invention envisions the use of a pharmaceutical composition comprising a LRP5 depleting agent for use in the methods of the invention.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions are formulated using one or more pharmaceutically acceptable excipients or carriers. In one embodiment, the pharmaceutical compositions comprise a therapeutically effective amount of LRP5 depleting agent and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

### Administration/Dosing

The regimen of administration may affect what constitutes an effective amount. For example, the therapeutic formulations may be administered to the patient either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

### Routes of Administration

One skilled in the art will recognize that although more than one route can be used for administration, a particular route can provide a more immediate and more effective reaction than another route.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

### Immune Response Stimulation.

In one embodiment, the invention comprises methods for providing anti-tumor immunity and for stimulating T-cell mediated immune response by administering the to the subject an effective amount of an inhibiting agent that blocks the interaction between DKK2 and LRP5. In another embodiment, the invention comprises methods for providing anti-tumor immunity and for stimulating T-cell mediated immune response by administering the to the subject an effective amount of a LRP5 antibody or fragment thereof that inhibits or reduces LRP5 expression or activity, with a pharmaceutical acceptable carrier.

The activation T lymphocytes (T cells) and its use within immunotherapy for the treatment of cancer and infectious diseases, is well known in the art (Melief et al., Immunol. Rev., 1995, 145:167-177; Riddell et al., Annu. Rev. Immunol., 1995, 13:545-586). As disclosed in the current invention, elimination of LRP5 leads to an activation of CD8+ cytotoxic T lymphocytes (CTL) and suppression of tumors.

Markers for CTL activation can be, but are not limited to, cytotoxins such as perforin, granzymes, and granulysin, cytokines, IL-2, IL-4, IFN-γ, PD-1, CD25, CD54, CD69, CD38, CD45RO, CD49d, CD40L, CD107a, CD137, CD134, CD314. The measurement in a sample of level of at least one of these markers can be used to assess CTL activation as presented herein the Examples section. Sorting of T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

The activation of Natural Killer cells (NK cells) and its use within immunotherapy for the treatment of cancer and infectious diseases, is well known in the art (Crouse, J. et al., 2015, Trends Immunol, 36: 49-58; Marcus, A., et al., 2014, Adv Immunol 122: 91-128; Palucka, A., et al., 2016, Cell 164: 1233-1247). As disclosed in the current invention, elimination of LRP5 leads to an activation of Natural Killer cells (NK) and suppression of tumors.

Markers for NK cell activation could be, but are not limited to, cytotoxins such as perforin, granzymes, and granulysin, cytokines, IL-2, IL-4, IL-15, IFN-γ, MHC-I haplotypes, NKG2D ligands (RAE-1α-ε, MULT-1, and H60a-c), Fas, TRAILR1/2, PD-1, CD25, CD54, CD69, CD38, CD45RO, CD49d, CD40L, CD107a, CD137, CD134, or CD314. The measurement in a sample of level of at least one of these markers can be used to assess NK cell activation as presented herein the Examples section. Sorting of NK cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

### Angiogenesis

Angiogenesis is a normal and vital process in growth and development, as well as in wound healing and in the formation of granulation tissue. The normal regulation of angiogenesis is governed by a fine balance between factors that induce the formation of blood vessels and those that halt or inhibit the process. When this balance is destroyed, it usually results in pathological angiogenesis which causes increased blood-vessel formation. Pathological angiogenesis is a hallmark of cancer and various ischemic and inflammatory diseases (e.g. cardiovascular diseases). As tumors cannot grow beyond a certain size or spread without a blood supply, blocking tumor angiogenesis is an effective approach in anticancer therapy. Also the use of angiogenesis inhibitors, also referred to as anti-angiogenic agents, are known in the art as relevant for treating ischemic and inflammatory diseases.

### Treatment of Cancer

In some aspects of the invention, treatment of cancer may include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers may include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis.

### Methods of Measurement

Any method known to those in the art can be employed for determining the level of DKK2 or LRP5 expression. For example, a microarray can be used. Microarrays are known in the art and consist of a surface to which probes that correspond in sequence to gene products (e.g. mRNAs, polypeptides, fragments thereof etc.) can be specifically hybridized or bound to a known position. To detect at least one gene of interest, a hybridization sample is formed by contacting the test sample with at least one nucleic acid probe. A preferred probe for detecting DKK2 or LRP5 is a labeled nucleic acid probe capable of hybridizing to DKK2 or LRP5 mRNA respectively. The nucleic acid probe can be, for example, a full-length nucleic acid molecule, or a portion thereof, such as an oligonucleotide of at least 10, 15, or 20 nucleotides in length and sufficient to specifically hybridize under stringent conditions to the appropriate target. The hybridization sample is maintained under conditions which are sufficient to allow specific hybridization of the nucleic acid probe to a target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a gene in the test sample, the sequence that is present in the nucleic acid probe is also present in the mRNA of the subject. More than one nucleic acid probe can also be used. Hybridization intensity data detected by the scanner are automatically acquired and processed by the Affymetrix Microarray Suite (MASS) software. Raw data is normalized to expression levels using a target intensity of 150. An alternate method to measure mRNA expression profiles of a small number of different genes is by e.g. either classical TaqMan® Gene Expression Assays or TaqMan® Low Density Array-micro fluidic cards (Applied Biosystems). Particularly, this invention preferably utilizes a qPCR system. Non-limiting examples include commercial kits such as the PrimePCRPathways® commercially available from Bio-rad (Berkley, Calif.).

The transcriptional state of a sample, particularly mRNAs, may also be measured by other nucleic acid expression technologies known in the art. mRNA can be isolated from the sample using any method known to those in the art. Non-limiting examples include commercial kits, such as the RNeasy® commercially available from Qiagen (Netherlands) or the Mini Kit the TRI Reagent® commercially available from Molecular Research Center, Inc. (Cincinnati, Ohio), can be used to isolate RNA. Generally, the isolated mRNA may be amplified using methods known in the art. Amplification systems utilizing, for example, PCR or RT-PCR methodologies are known to those skilled in the art. For a general overview of amplification technology, see, for example, Dieffenbach et al., PCR Primer: A Laboratory Manual, Cold Spring Harbor Laboratory Press, New York (1995).

Another accurate method for profiling mRNA expression can the use of Next Generation Sequencing (NGS) including first, second, third as well as subsequent Next Generations Sequencing technologies.

In other aspects of the present invention, determining the amount or detecting the biological activity of a peptide, polypeptide can be achieved by all known means in the art for determining the amount of a peptide or polypeptide in a sample. These means comprise immunoassay devices and methods which may utilize labeled molecules in various sandwich, competition, or other assay formats. Such assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Mice.

ApcMin/+(C57BL/6J-ApcMin/J) and MX1Cre [B6.Cg-Tg(Mx 1-cre)1Cgn/J] mice were acquired from Jackson Laboratory. Wildtype C57BL/6 mice were purchased from Envigo (Harlan). The LoxP-floxed Lrp5 (Lrp5f/f) and Lrp6 (Lrp6f/f) mice were obtain from Bart Williams (54). The Lrp5f/f and Lrp6f/f mice were backcrossed to C57/BL6 for more than 7 generations before being intercrossed with MX1Cre. LRP5 and LRP6 gene disruption was induced by intraperitoneal injection of the Lrp5fl/flMX1Cre mice with 40 μl poly-I:C (10 mg/mL) every other day for 4 treatments. The mice were used for NK cell isolation three weeks after the poly-I:C treatment. For adoptive bone marrow transfer, bone marrows from the Lrp5fl/flMX1Cre mice were transferred to lethally irradiated C57/BL6 mice (8 weeks old) via retro-orbital injection. After recovery (8 weeks), the mice were treated with poly-I:C and used in experiments three weeks after poly-I:C treatment.

### Antibodies.

Antibodies to phospho-Stat5 (Tyr694) (CST, 4322s), LAMP1 (sc-19992, Santa Cruz), EEA1 (BD Bioscience, 612006), phospho-AKT (serine 473) (CST, 4060), AKT1 (CST, 9272), phospho-ERK1/2 (Thr202/Tyr204) (CST, 4377), ERK1/2 (CST 9102), perforin (CST, 3693), granzyme B (CST, 4275), β-actin (CST, 3700), FLAG (Sigma Aldrich, F3165), β-catenin (BD Bioscience, 610153), LRP5 (CST, 5731), LRP6 (CST, 3395), mouse CD4-PE (eBioscience, 12-0042-82), mouse NK1.1-APC (BioLegend, 108710), mouse CD8a-PE-Cyanine7 (eBioscience, 25-0081-82), mouse CD69-PE (Biolegend, 104508), human/mouse granzyme B-FITC (BioLegend, 515403), mouse CD314 (NKG2D)-PE-Cyanine7 (eBioscience, 25-5882-81), mouse CD3e-PE (eBioscience, 12-0031-82), mouse IFNγ-PE (eBioscience, 12-7311-81), CTLA-4/CD152 (1B8)-FITC (Thermo Fisher, HMCD15201), human CD45-eFluor® 450 (eBioscience, 48-0459-41), mouse CD107a-V450 (BD, 560648), mouse CD8a-APC (eBioscience, 17-0081-81), mouse CD25-Alexa Fluor® 488 (eBioscience, 53-0251-82), mouse CD279 (PD-1)-PE (BioLegend, 135205), Ki67 (Abcam ab, 15580), Cleaved Caspase-3 (Asp175), (CST, 9661S), CD31 (Abcam ab, 28364), Fluorescein (FITC)-labeled AffiniPure F(ab′)₂ Fragment Donkey Anti-Mouse IgG (H+L) (Jackson lab, 715-096-151), Mouse Integrin alpha 4 beta 7 (LPAM-1) APC (eBioscience, 17-5887-80), Human CD56 (NCAM) APC (eBioscience, 17-0566-41), Human CD16 PE (eBioscience, 12-0167-42), Human CD3 eFluor® 450 (eBioscience, 48-0037-42), and Alexa Fluor® 647-labeled AffiniPure F(ab′)₂ Fragment Goat Anti-Rabbit IgG (H+L) (Jackson lab, 111-606-045). Mouse monoclonal antibody to DKK2 (5F8) was generated using standard hybridoma technology through immunization of mice with a synthetic peptide (KLNSIKSSLGGETPGC; SEQ ID NO: 21) of human DKK2 at AbMax (Beijing, China). The heavy and light chain peptide sequences of 5F8 are the following: GAELVRPGASVKLSCKASGYSFTNYWMNWVKQRPGQGLEWIGMIHPSDSETRLNQ KFKDKATLTVDKSSSTAYMQLSSPTSEDSAVYYCAREGRLGLRSYAMDYWGQGTS VTVSS (SEQ ID NO: 22), and PSSLAMSVGQKVTMSCKSSQSLLNSSNQKNYLAWYQQKPGQSPKLLVYFASTRESG VPDRFVGSGSGTDFTLTITSVQAEDLADYFCQQHYITPLTFGAGTKLE (SEQ ID NO: 23) respectively. Other mouse monoclonal antibodies to DKK2 could also be used for this invention, such as but not limited to, antibody 1A10 generated using standard hybridoma technology through immunization of mice with a synthetic peptide (CKVWKDATYSSKAR; SEQ ID NO: 24) of human DKK2 at AbMax (Beijing, China). The heavy and light chain peptide sequences of 1A10 are the following: LQQSGPELVKPGASVKISCKASGYSFTGYFVNWVKQSHGKSLDWIGRIIPYNGDTFY NQKFKGKATLTVDKSSTTAHMELLSLTSEDSAVYYCGRGDYWGQGTSVTVSS (SEQ ID NO: 25), and PLTLSVTIGQPASISCKSSQSLLDSDGKTYLNWLLQRPGQSPKRLIYLVSKLDSGVPDR FTGSGSGTDFTLKISRVEAEDLGVYYCWQGTHFPQTFGGGTKLEIK (SEQ ID NO: 26) respectively. Therapeutic anti-PD-1 antibodies are hamster mAb clone G4 (Hirano, F. et al. Cancer Res. 65, 1089-1096 (2005)) and Clone J43 (BioXcell, BP0033-2) with polyclonal Armenian Hamster IgG (BioXcell, BE0091) as the control IgG.

### Quantitative RT-PCR.

Total RNAs were isolated from cells using the RNeasy Plus Mini Kit (QIAGEN). Complementary DNAs were synthesized from the RNAs using the iScript cDNA Synthesis Kit (Bio-Rad). Quantitative PCR was done using the iTaq Universal SYBR Green Supermix (Bio-Rad). The primer sequences are listed in FIG. 16 (SEQ ID NOs: 1-4).

### ELISA.

Recombinant mouse DKK2 or DKK1 protein (20 ng/ml, R&D) in a blocking buffer (1% BSA in PBS) was incubated in a 384-well microtiter plate for overnight at 4° C. The plate was washed with PBS twice and incubated with the blocking buffer for one hour at room temperature. The plate was then incubated with the anti-DKK2 5F8 antibody in the blocking buffer for 1 hour at room temperature. After repeatedly washing, the plate was incubated with an HRP-conjugated secondary antibody for 1 hour at room temperature. A chemiluminance substrate (Thermo Fisher 37070) was added to the plate, and the plate was read by an EnVision plate reader.

### DKK2-AP Binding Assay.

The binding assay was performed as previously described (56). In brief, HEK293T cells were transfected with LacZ or LRP5 using Lipofectamine Plus for 24 hours. Cells were washed once with a cold washing buffer (Hanks' buffered salt solution containing 1% bovine serum albumin, 20 mM HEPES, and 0.5% NaN3) and incubated with the washing buffer containing 20% of DKK2-AP conditioned medium on ice for 2 h. The cells were then washed three times with the washing buffer and lysed with 1% Triton X-100 and 20 mM Tris-HCl, pH 7.5. The lysates were heated at 65° C. for 10 min to inactivate endogenous AP and then added with a chemiluminescence AP substrate (Thermo Fisher T1015). The activity was measured by an EnVision plate reader.

### Tumor Graft.

MC38 or YUMM1.7 melanoma tumor cells (0.5-1×106) were mixed with BD Matrigel (Matrix Growth Factor Reduced) (BD 354230) in 100 μl and inoculated subcutaneously at the right flanks of the backs of female C57/BL mice (8-10 weeks old). Tumor growth was measured by calipers, and size was expressed as one half of the product of perpendicular length by square width in cubic millimeters. For antibody treatment, control IgG3 antibody and anti-DKK2 antibody were diluted in PBS, and 100 μl was injected i.p. at intervals indicated in the Figures. For survival tests, mice are euthanized when the tumor size exceeding 1800 mm3 for MC38 and 1200 mm3 for YUMM1.7.

### Preparation of Tumor Infiltrating Leukocytes.

Tumors were minced using scissors and scalpel blades and incubated with a digestion buffer [RPMI1640, 5% FBS, 1% PS, 25 mM HEPES and 300 U collagenase (Sigma C0130)] in a shaker for 2 h at 37° C. Disperse cells were filtered through a 70 μm cell strainer to eliminate clumps and debris. After centrifugation for 5 minutes (500×g) at 4° C., cell pellets were resuspended in the Red Blood Cell Lysis Buffer (Sigma R7757) and incubated at RT for 5 min to remove erythrocytes. Cells were pelleted again, resuspended and incubated in 0.05% Trypsin/EDTA at 37° C. for 5 min, followed by DNA digestion with the addition of Type I DNase (1 μg/ml final, Sigma D4263) for 5 min. Trypsin digestion was stopped by the addition of FBS to 5%, and cells were filtered again by a 40 μm cell strainer. Finally, the cells were pelleted again and resuspended in PBS at a concentration of 2×107.

### Flow Cytometry.

Cells in single cell suspension were fixed with 2% PFA (Santa-Cruz sc-281692). After washing with a Flow Cytometry Staining Buffer (eBioscience 00-4222-26), cells were stained with antibodies for cell surface markers for 1 hour on ice in the dark. For staining of intracellular proteins, the cells were washed and resuspended in the Permeabilization Buffer (BD 554723) and stained by antibodies in the Permeabilization Buffer for 1 hour on ice in the dark. The cells were then pelleted and resuspended in the Flow Cytometry Staining Buffer for flow cytometry analysis.

### Tumor Sectioning and Immunostaining.

Tissues were fixed with 4% PFA (Santa-Cruz sc-281692) for 4-6 hours on a shaker at 4° C. They were then washed with PBS three times and perfused in 20% sucrose solution in PBS overnight at 4° C. They were subsequently mounted in the OCT embedding compound and frozen first at −20 and then at −80° C. Tissue sections were prepared at 8 μm thickness using a cryostat and mounted onto gelatin-coated histological slides, which were stored at −80° C. For immunostaining, slides were thawed to room temperature and fixed in pre-cold acetone for 10 minutes, followed by rehydration in PBS for 10 minutes. The slides were incubated in a blocking buffer (1% horse serum and 0.02% Tween 20 in PBS) for 1-2 hours at room temperature, followed by incubation with primary antibodies, which were diluted in an incubation buffer (1% horse serum, 0.02% Tween 20 in PBS), overnight at 4° C. The slides were then washed three times with PBS and incubated with a secondary antibody [donkey anti-rabbit IgG H&L (DyLight® 550) preadsorbed (abcam ab96920)] in the incubation buffer for 1 hour at room temperature. After repeated washes the slides were mounted with an antifade mounting media containing DAPI (Thermo Fisher P36931) and visualized using a confocal microscope.

### Effector Immune Cell Depletion.

For depletion of NK cells, the anti-NK1.1 (PK136, BioXcell BE0036) or isotype control (BioXcell BE0085) was injected i.p. at 300 ug/mouse at Day −1, 5, 11 and 17 of tumor cell inoculation. For CD8 depletion, the anti-CD8α (YTS 169.4, BioXcell BE0117) or isotype control (Clone LTF-2, BioXcell BE0090) was injected i.p. at 300 ug/mouse at Day 12, 15 and 19 of tumor cell inoculation.

### Preparation and Treatment of Mouse Primary NK, CD8+ and IEL Cells.

Mouse primary NK and CD8+ T cells were isolated from the spleens by using the NK cell and CD8+ T cell isolation kits according to the manufacturer's instructions (Miltenyi Biotec #130-090-864 and #130-104-075), respectively. Primary NK cells were cultured in RPMI-1640 (Gibco, 11875-093) supplemented with 10% FBS, penicillin (100 U/ml), streptomycin (100 μg/ml), 2-mercaptoethanol (500 μM) and HEPES (10 mM) at 37° C. supplemented with 5% CO2 in the presence of recombinant murine IL-15 (50 ng/ml) for 24 hours before treatment with DKK2, CHIR99021, or WNT3A. CD8+ T cells were cultured in the same culture medium and condition as NK cells, but supplemented with IL-15 (200 ng/ml) and IL-15 Rα (1 μg/ml recombinant Mouse IL-15 receptor alpha Fc chimera Protein from R&D) for 96 hours before DKK2 treatment. Mouse primary intraepithelial lymphocytes (IELs) were prepared as described in (Little et al., The Journal of Immunology 175, 6713-6722 (2005) and Li et al. Infect Immun 80, 565-574 (2012)). In brief, the small intestine was everted, divided into four pieces, and washed twice in phosphate-buffered saline (PBS) containing 100 U/ml penicillin/streptomycin The specimens were then incubated with stirring at 37° C. in prewarmed Ca2+ and Mg2+-free Hanks' solution containing 100 U/ml penicillin-streptomycin, 5% fetal calf serum (FCS), 2 mM dithiothreitol (DTT), and 5 mM EDTA for 30 min, followed by vigorous shaking for 30 s. The supernatants were passed over two nylon wool columns to remove undigested tissue debris. The lymphocytes obtained were pooled and enriched on a discontinuous (40% and 70%) Percoll density gradient. Cells at the interface between the 40% and 700%, fractions (IELs) were collected for treatment with IL-15 (200 ng/ml) and DKK2 (200 ng/ml), followed by flow analysis.

### Preparation of Human NK Cells.

Peripheral blood mononuclear cells from normal humans were purchased from ZenBio (SER-PBMC-200). Human NK cells were isolated from the PBMCs by using the human NK cell isolation kit according to the manufacturer's instruction (Miltenyi Biotec #130-092-657). Human NK cells were cultured in RPMI-1640 (Gibco, 11875-093) supplemented with 10% heat-inactivated FBS, penicillin (100 U/ml), streptomycin (100 μg/ml), 2-mercaptoethanol (500 μM) and HEPES (10 mM) at 37° C. supplemented with 5% CO2 in the presence of recombinant human IL-15 (50 ng/ml) before treatment with recombinant human DKK2 protein.

### NK and Tumor Cell Co-Culture.

Primary NK cells were isolated from the spleens as described above and cultured in the presence of 50 ng/ml recombinant murine IL-15 for 24 hours. Meanwhile, tumor cells were plated in the 96 well plate for overnight. NK cells were added into the tumor cells at 7:1 radio in the presence of the IgG3 antibody or anti-DKK2 5F8 for 9 hours at 37° C. For testing the effect of DKK2 in co-culture, isolated NK cells were cultured in the presence of 50 ng/ml recombinant murine IL-15 for 24 hours and then cultured in the presence or absence of DKK2 for another 24 hours before the NK cells were added to pre-seeded MC38 cells at 7:1 (NK:MC38) radio. The numbers of live tumor cells were determined by a Guava flow cytometer (EMDmillipore), whereas the cell apoptosis was assessed by flow cytometry using an Annexin V apoptosis detection kit (eBioscience, 88-8007).

### Immunocytostaining.

Primary NK cells were prepared as described above and treated as indicated in the Figures. They were then placed on poly-lysine coated coverslips and incubated for 30 min at room temperature. HEK293T cells grown on coverslips were transfected and stimulated as indicated in the Figures. Cells were fixed with 4% PFA for 10 min at room temperature and permeabilized with ice-cold methanol for 10 min at −20° C. After rinsed with PBS for 3 times, cells were blocked with a blocking buffer (5% normal donkey serum and 0.5% triton in PBS) for 1 hr at room temperature. Primary antibodies were then diluted in PBS with 0.5% BSA and applied to cells with overnight incubation at 4° C. Cells were rinsed with PBS for 3 times and incubated with diluted fluorochrome-conjugated secondary antibodies (in PBS with 1% BSA) for 1 hr at room temperature. Finally, cells were rinsed with PBS for 3 times and mounted with Prolong Gold Antifade solution (Thermo Fisher) for confocal microscopy.

### Immunoprecipitation.

293T cells were transfected with plasmids encoding STAT5 and/or LRP5C-Flag with Lipofectamine Plus. The cells were lysed 24 hours after transfection in the lysis buffer [50 mM HEPES (pH 7.4), 150 mM NaCl, 1% Triton X-100, 10% glycerol, 2 mM MgCl2, 2 mM EGTA] with the protease inhibitors cocktail (Roche) and phosphatase inhibitors (Phospo-stop from Roche) on ice. Cell lysates were centrifuged to remove insoluble materials. Immunoprecipitation was performed with an anti-Flag antibody for overnight, followed by 2-hour incubation of Protein-A/G Plus beads (Santa Cruz), at 4 degree. The beads were washed repeatedly, and bound proteins were analyzed by Western blotting.

### Reporter Gene Assays.

The Stat5 reporter assays were done in HEK293T cells for activated JAK1-induced activity or in those stably expressing JAK3, IL2/15Rβ and the common receptor γ subunit for IL15 induced activity. Cells were seeded at 8×104 cells per well in 48 well plate. The next day, cells were transfected by Lipofectamine 2000 (Invitrogen) with the pGL4.52-STAT5-Luciferase (Promega) and tagRFP (internal control) plasmids together with other plasmids expressing genes of interest. The total amount of plasmid was kept at 125 ng per well. The cells were added 24 hours after transfection with IL15/IL15Rα-Fc complex or mock. Six hours later, the cells were lysed and subjected to RFP fluorescence and luciferase luminescence measurement using an Envision Multilabel plate reader. The reporter gene activity is shown after being normalized against RFP readings. The LEF reporter assay was carried out in HEK293 cells that were transfected with the TOPFlash and GFP plasmids. The rest is the same as above. The reporter gene activity is shown after being normalized against GFP readings.

### Generation APC Mutant Cells.

Gene editing of the APC genes in MC38 and HCT116 cells was done using the CRISPR-Cas9 system as previously described (Ran et al., Nat. Protoc 8, 2281-2308 (2013)). The cells were transfected with two Cas9 plasmids expressing two guiding RNAs targeted to the APC gene. This will lead to a deletion of the gene and a frameshift of the APC gene. As these two guiding RNAs were coexpressed with GFP or RFP, respectively, the GFP+RFP+ cells were sorted directly into 96 well plates at the density of 1.2 cells/well. Homozygous deletions of APC were detected by PCR and confirmed by DNA sequencing. Positive clones were pooled to avoid clonal effects. The guiding and PCR sequences are listed in FIG. 16.

### LRP Internalization Assay.

HEK293 cells were treated with mock or recombinant mouse DKK2 protein (4 nM) in culture medium for duration indicated. The cells were washed with pre-cold PBS and cell surface proteins were biotinylated with 0.5 mg/ml of EZ-Link Sulfo-NHS-SS-Biotin (Thermo Fisher, 21445) in a PBS buffer on ice for 30 min. The reaction was stopped by addition of PBS containing ice-cold 50 mM NH4Cl, followed by repeated washes with ice-cold PBS. The cells were then lysed with in a buffer containing 1.25% Triton X-100, 0.25% SDS, 50 mM Tris HCl PH8.0, 150 mM NaCl, 5 mM EDTA, 5 mg/ml iodoacetamide, 10 ug/ml PMSF, and the Roche proteinase inhibitor cocktail. After centrifugation, aliquots were taken as lysate controls, and the rest of supernatants were used in pull-down with NeutrAvidin beads (Thermo Fisher, 29200), followed by analysis by Western blotting.

### RNA Sequencing and Data Analysis.

Primary NK cells were isolated from the spleens as described above and cultured in the presence of 50 ng/ml recombinant murine IL-15 for 24 hours and then cultured in the presence or absence of 10 nM DKK2 for another 24 hours before mRNA was isolated and purified by using RNeasy Plus Mini Kit (Qiagen). RNA-seq libraries were prepared using the TrueSeq Stranded Total RNA Library Prep Kit (Illumina) and sequenced on Illumina HiSeq 2500 with 50 base single end read Gene expression analysis was prepared and accessed as described previously (Trapnell et al., Nat Protoc 7, 562-578 (2012) with GENCODE annotation Ml). Pathway analysis of RNA sequencing results were performed at www.amp.pharm.mssm.edu/Enrichr/enrich. Gene enrichment analysis was performed with Motif Gene Set (software.broadinstitute.org/gsea/msigdb/index.jsp) (Subramanian et al., PNAS 102, 15545-15550 (2005)).

### Correlation of DKK2 Expression and Patient Survival.

The DKK2 expression, overall survival, and relapse-free survival data were obtained from the TCGA provisional datasets as of Jul. 20, 2016. The high and low DKK2 expressers were grouped using an arbitrary cutoff percentile of 15%. The Mantel-Cox Log-Rank tests were done using the GraphPad Prism 7 software.

The results of the experiments are now described in the following examples.

### Example A: Loss of APC Drives DKK2 Expression

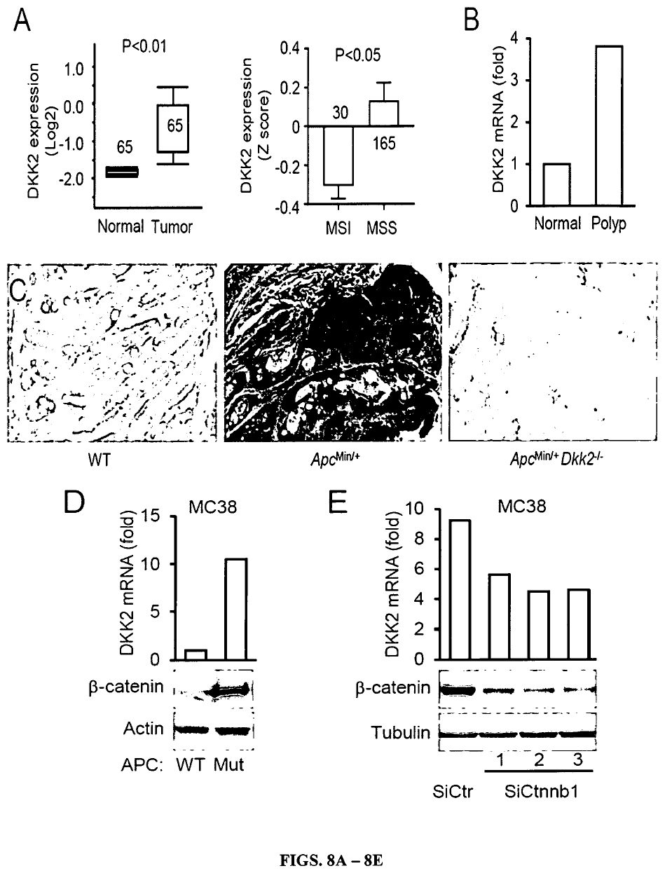

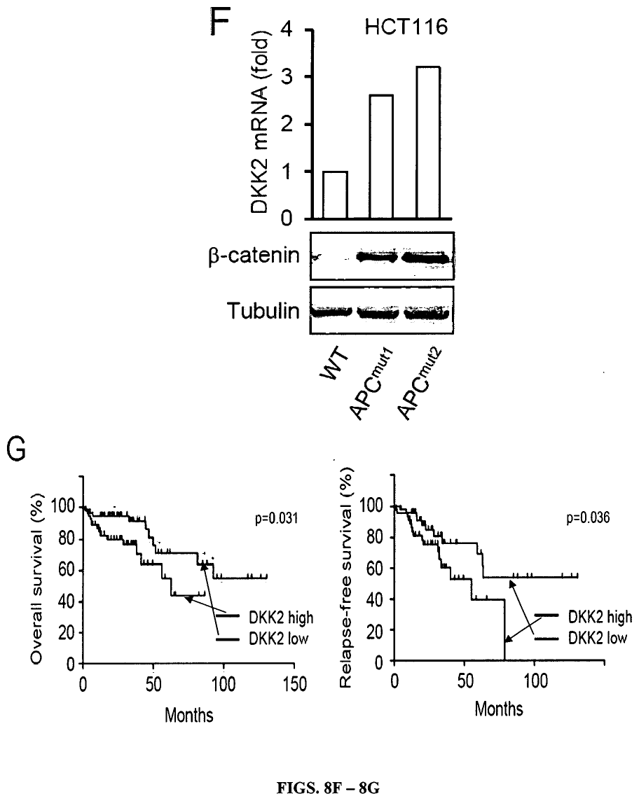

Analysis of the Gaedcke cohort (Gaedcke et al., Genes Chromosomes Cancer 49, 1024-1034 (2010)) in the Oncomine database (www.oncomine.org) revealed that DKK2 expression was significantly upregulated in human CRC samples than the non-tumorous colorectal tissues (FIG. 8A). This observation is consistent with the finding of a previous report (Matsui et al., Cancer Sci 100, 1923-1930 (2009)). In addition, DKK2 expression in microsatellite-stable (MSS) CRCs, more than 80% of which harbor APC mutations, is significantly higher than that in microsatellite-instable (MSI) CRCs based on the analysis of the dataset reported in The cancer Genome Atlas Network (Nature 487, 330-337 (2012)) (FIG. 8A). Examination of DKK2 mRNA contents in the polyps isolated from the intestines of the Apc^{Min/+} mice indicated that DKK2 was expressed at about four times higher level than in normal intestines (FIG. 8B). The Apc^{Min/+} mice harbor a mutation in one of the Apc alleles and produce frequent intestinal tumors due to spontaneous loss of the wildtype allele (Su et al., Science 256, 668-670 (1992)). Moreover, immunostaining of the DKK2 protein confirmed the upregulation of DKK2 expression in the polyps from the Apc^{Min/+} mice (FIG. 8C). To test if the loss of APC drives DKK2 expression via the Wnt-p-catenin pathway, the Apc gene was mutated in the MC38 cells by using the CRISPR/Cas9 technology to cause homozygous C-terminal deletion of the APC protein starting at Gly-855 and observed that DKK2 expression was markedly upregulated in the APC-null MC38 cells (FIG. 8D). This upregulation of DKK2 expression could be suppressed by P-catenin siRNAs (FIG. 8E), suggesting the involvement of p-catenin in driving the DKK2 expression. The APC gene was also mutated in the HCT116 human colon cancer cells by introducing homozygous C-terminal deletions of the APC protein starting at Gly-857 and Ser-1346, respectively. Despite the presence of the stabilization mutation of one p-catenin allele in these cells, the APC mutations led to marked increases in DKK2 expression (FIG. 8F). Therefore, these results together indicate that the loss of APC can drive DKK2 expression in both mouse and human.

### Example 2: Blockage of DKK2 Suppresses APC-Loss-Induced Tumor Formation

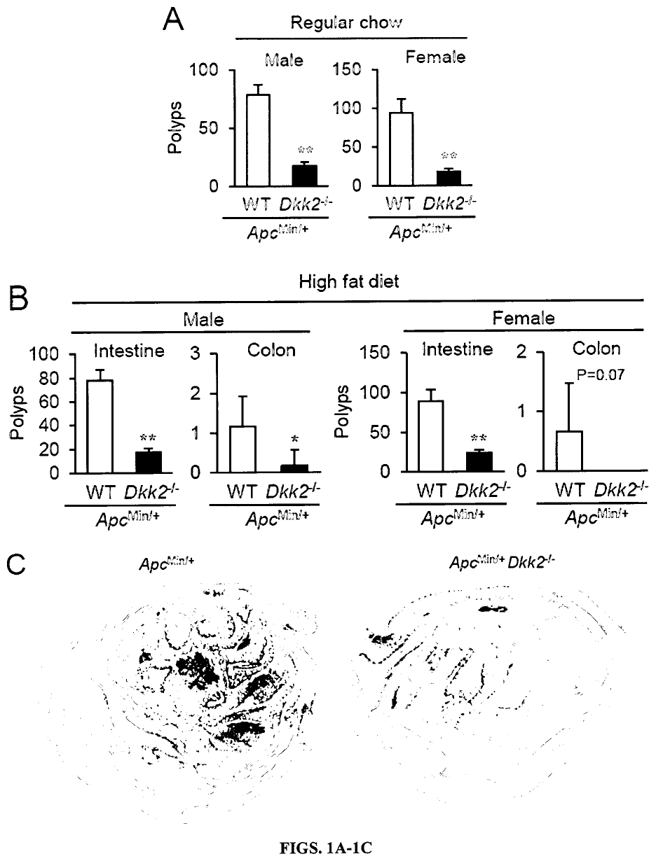

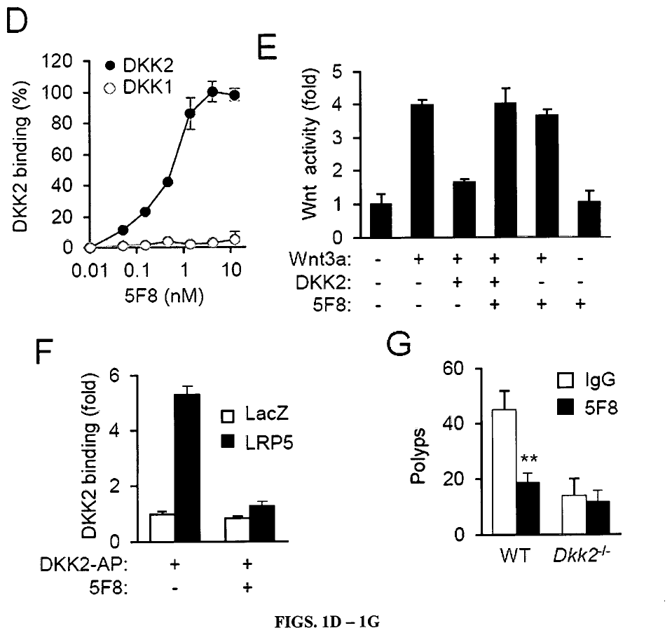

Analysis of the datasets of the TCGA CRC cohort revealed a significant correlation of high DKK2 expression with poor survival rates (FIG. 8G). This suggests that DKK2 may play an important role in CRCs. Given that DKK2 is a Wnt antagonist, the conventional wisdom is that inactivation of DKK2 might increase Wnt activity and hence lead to or accelerate cancer formation. To investigate the involvement of DKK2 in tumorigenesis, DKK2^{-/-} mice were observed for up to one year and there was no histologically discernable dysplasia in tissues, including the gastrointestinal tract. The role of DKK2 in tumorigenesis was further tested by examining the effects of DKK-2 deficiency on polyp formation in APC^{Min/+} mice (designated APC) and APC^{Min/+} DKK2^{-/-} (APCKO) mice. Mice were housed in a specific pathogen free vivarium and fed with regular or high fat chow. Intestinal sections were stained with methylene-blue and polyps were counted under a stereomicroscope. In the absence of DKK2, tumorigenesis was significantly reduced as indicated by lower number and size of intestinal polyps (FIGS. 1A and 1B). This phenomenon was seen in groups of male and female mice on both high and low fat diets with consistent results. There were smaller and fewer intestinal polyps in an APC^{Min/+} DKK2^{-/-} mice than in APC^{Min/+} mice, as shown in representative histological sections of intestine, stained with haematoxylin and eosin, from male mice fed regular chow (FIG. 1C). Together, these data strongly suggest that without DKK2-mediated signaling, progression of colon cancer is significantly reduced.

A functional mouse monoclonal anti-DKK2 antibody (5F8) was developed to specifically target and neutralize DKK2, but to not cross react with DKK-1. ELISA data demonstrate that the 5F8 antibody bound specifically to DKK2 antigen in a dose-dependent manner (FIG. 1D). It has been shown that DKK2 as well as other DKK family proteins, inhibit canonical Wnt signaling by binding to Wnt co-receptor LRP 5/6 with high affinity and competing with Wnt molecules for receptor binding (MacDonald, B., et al., Dev Cell, 2009. 17(1): p. 9-26; Bao, J., et al., Sci Signal, 2012. 5(224): pe22). To determine if 5F8 diminished DKK2 inhibition of Wnt signaling, a Wnt reporter gene assay was used to measure Wnt activity. HEK293 cells were transfected with Wnt reporter gene TOPFlash and Wnt reporter activity was examined. As shown in FIG. 1E, Wnt3a increased Wnt reporter activity, but addition of DKK2 with Wnt3a inhibited Wnt signaling. Taken together, the data indicate that 5F8 mediates an antitumorigenic response via Wnt co-receptor LRP 5/6 activity.

To examine whether 5F8 blocks DKK2 binding to LRP5, a binding assay was performed. In this study, HEK293 cells were transfected with LacZ (control plasmid) or LRP5 expression plasmid. The direct binding of DKK2-AP fusion protein to LRP5 overexpressed on the cell surface was measured, both in the presence or absence of 5F8. As shown in FIG. 1F, the 5F8 antibody blocked DKK2 binding to LRP5.

To investigate if the 5F8 antibody reduced polyp formation in APC^{Min/+} mice, similar to DKK2 deficiency in APC^{Min/+} DKK2^{-/-} mice, the tumor burden was analyzed in treated and control mice. Treatment of APC^{Min/+} mice with 5F8 antibody for 8 weeks significantly diminished the number of intestinal polyps when compared to untreated mice. Further, the number of polyps in 5F8-treated APC^{Min/+} mice was essentially the same as the number of polyps found in APC^{Min/+} DKK2^{-/-} mice, either 5F8 or control IgG treated mice (FIG. 1G). Taken together, the results provide proof of principle evidence that 5F8 is a blocking antibody for DKK2 that suppresses tumor formation via the Wnt co-receptor LRP 5/6 pathway. Further, 5F8 blocks the interaction between DKK2 and LRP5 interaction, but in doing so, 5F8 also promoted DKK2-mediated disinhibition of Wnt signaling.

### Example 3: DKK2 Blockade Modulates Tumor Immune Microenvironment

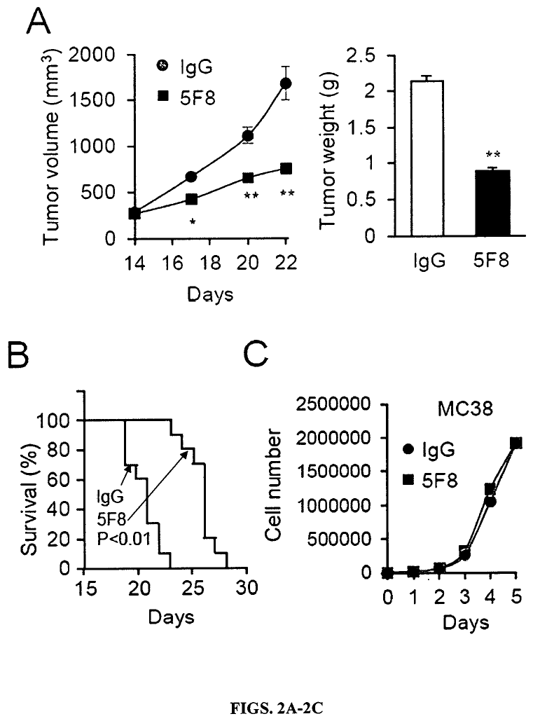

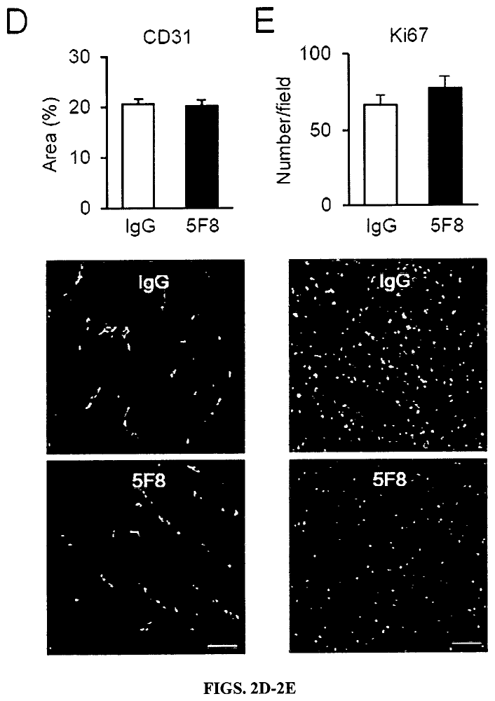

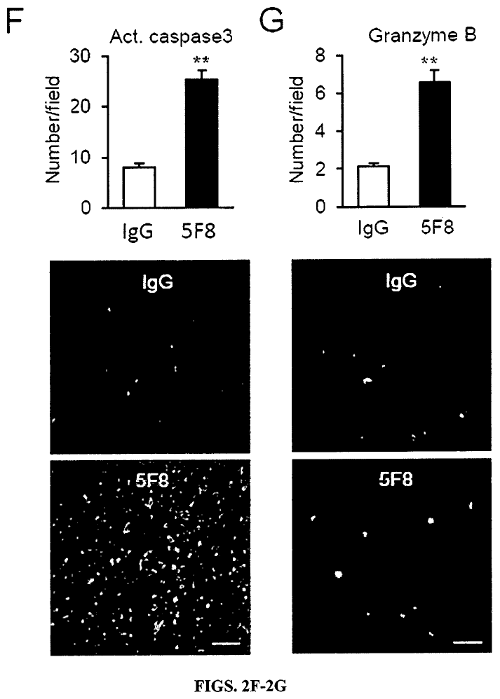

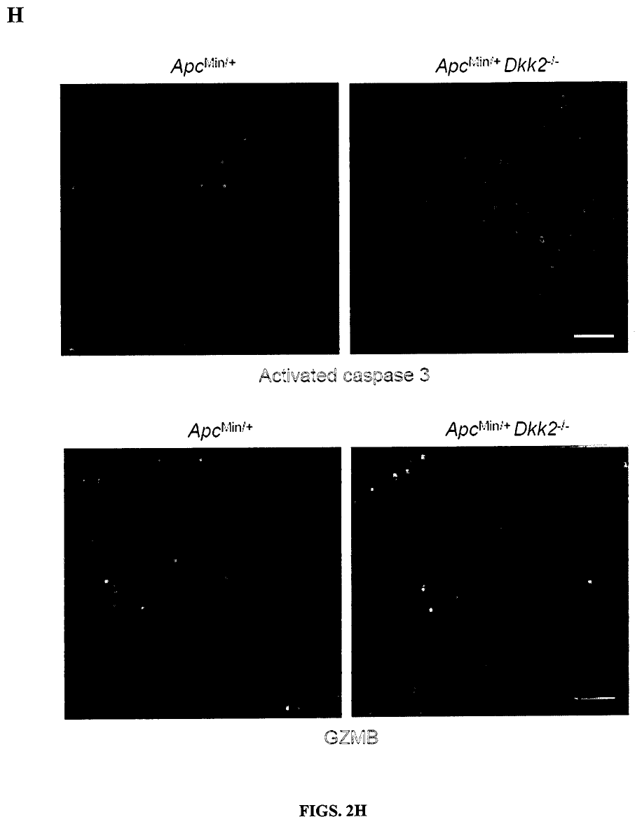

MC38 cells, which were derived from mouse colon carcinoma in a C57BL mouse, progress very fast when grafted to immunocompetent WT C57BL mice. Thus, this allograft model, also known as a syngeneic model, can be used to test the therapeutic potential of the anti-DKK2 antibody 5F8 in vivo with a functional host immune system. In one study, C57BL mice (10 week old female mice, n=5 per group) were grafted with MC38 cells via the subcutaneous (s.c.) route. Fourteen days later, the mice were treated every 3 days via the intraperitoneal (i.p.) route with mouse IgG or 5F8 (8 mg/kg). Tumors were collected on Day 22 and weighed. FIGS. 2A-2B show that treatment with 5F8 significantly inhibited the tumorigenic growth of subcutaneously grafted MC38 cells in C57BL mice when compared to control antibody (IgG3) treatment. These results suggest that, while the MC38 cells retain functional APC (FIGS. 8D-8E), they express a sufficient amount of DKK for anti-DKK treatment to work. This syngeneic cancer model was thus used to determine the mechanism by which DKK2 blockade suppresses tumor progression as the use of genetic tumor models are very time-consuming and costly. Because 5F8 did not affect the growth of MC38 cells in culture (FIG. 2C), the antibody might impede tumor progression by altering the tumor microenvironment. If blockade of DKK2 signaling does not reduce tumor cell growth, then anti-DKK2 signaling mediates tumor suppression by another route.

To test the effects of 5F8 antibody on tumor cell microenvironment such as changes in angiogenesis, proliferation or apoptosis, MC38 tumors were examined using an immunohistological approach. In this study, C57BL mice (10 week old female mice, n=5 per group) were grafted with MC38 cells via the subcutaneous (s.c.) route. Fourteen days later, the mice were treated every three days via the intraperitoneal (i.p.) route with mouse IgG or 5F8 (10 mg/kg). Visualization of MC38 tumors for CD31 expression, an extracellular protein involved in angiogenesis, showed no significant difference in angiogenesis in treated or control (IgG) tumors (FIG. 2D; graph and representative images). Histological analysis of MC38 tumors exposed to 5F8 or control (IgG) treatment for Ki67 expression, a protein associated with cell proliferation, also showed no significant difference in tumor cell proliferation (FIG. 2E). To test if DKK2 expression may alter the tumor microenvironment to induce apoptosis via proper anti-tumor immune response, the levels and anti-tumor activity of cytotoxic effector immune cells were measured. Immune cytotoxic cells, such as natural killer cells (NK) and CD8+T lymphocytes, are capable of directly killing tumor cells by secreting preformed granules containing perforin and granzymes. Uptake of granzyme B (gzmb), which is a serine protease, induces target cell apoptosis via a pathway involving proteolytic activation of Caspases, cleavage of Bid, and fragmentation of DNA. (Thornberry et al., J Biol Chem, 1997. 272(29): p. 17907-11; Heusel et al., Cell, 1994. 76(6): p. 977-87). Histological analysis of MC38 tumors revealed significant increases in the number of granzyme B-positive cells in 5F8 treated cells over control (IgG) cells (FIG. 2G). Further, visualization of MC38 tumors for cleaved caspase 3 [Activated Caspase 3 (Casp 3)], a marker for cells induced to die via an apoptotic pathway, showed a significant enhancement of apoptosis in 5F8 treated cells over control (FIG. 2F). Taken together, the data demonstrate that DKK2 blockade by 5F8 upregulates apoptosis within tumor cells [or tumors], without modifying tumor cell proliferation or angiogenesis within the tumor cell microenvironment. Further, the data suggest that blocking LRP5-signals mediated by DKK2 could upregulate apoptosis in tumor cells and an LRP5-specific inhibition may enhance levels of tumor cell apoptosis, without altering angiogenesis or tumor cell proliferation.

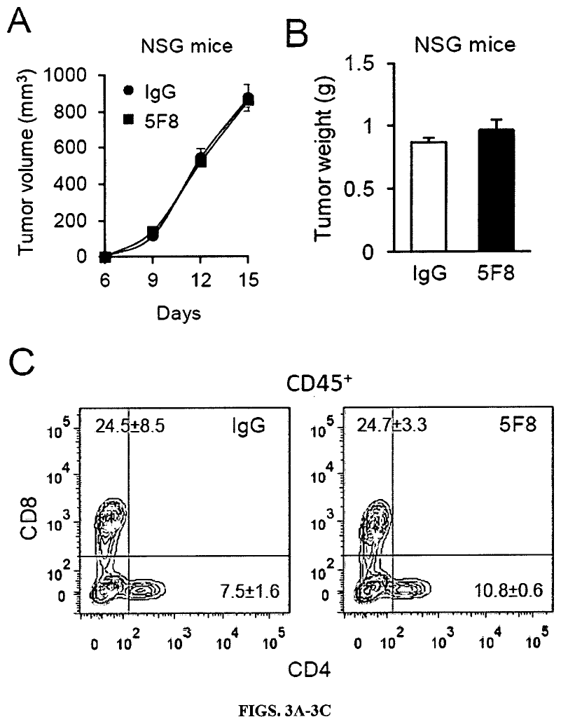

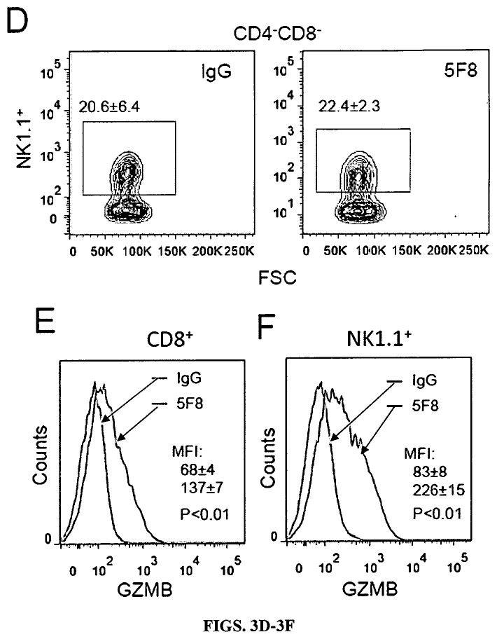

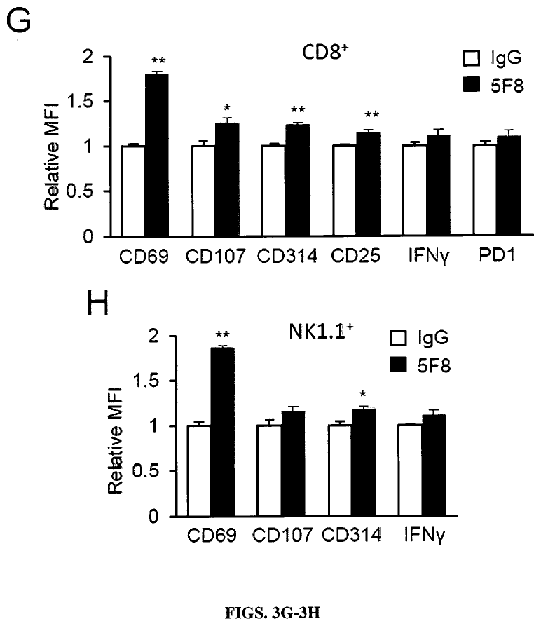

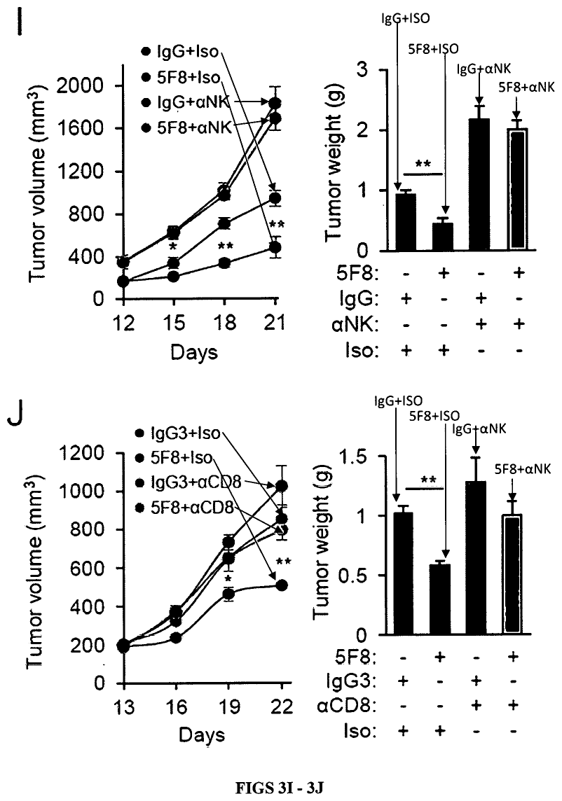

In addition, increases in apoptosis and granzyme B staining were also observed in the Apc^{Min/+} Dkk2^{-/-} polyps compared to the Apc^{Min/+} ones (FIG. 2H). Granzyme B is largely produced by cytotoxic immune cells including natural killer (NK) and CD8+ T cells and induces target tumor cell apoptosis (Afonina et al., Immunol Rev 235, 105-116 (2010)). Thus, the above data suggest that DKK2 blockade may act through the modulation of the immune microenvironment. Consistent with this conclusion, when the MC38 cells were grafted onto the immunodeficient NSG mice, which lack mature leukocytes including NK cells and cytotoxic T lymphocytes, 5F8 failed to show its tumor suppressive effect (FIGS. 3A-3B). Taken together these data indicate that minimizing DKK2 signaling via the Wnt co-receptor LRP5/6 substantially suppresses tumor growth and enhances animal survival. Blocking LRP5-mediated DKK2 signaling, without modifying Wnt signaling, would provide an effective therapeutic to increase apoptosis of tumor cells. Further, the data suggest that treatment of animals with an LRP5-specific antibody could enhance both the tumor suppressive effects and animal survival.

### Example 4: DKK2 Blockade Enhances NK and CD8+ Cell Activation

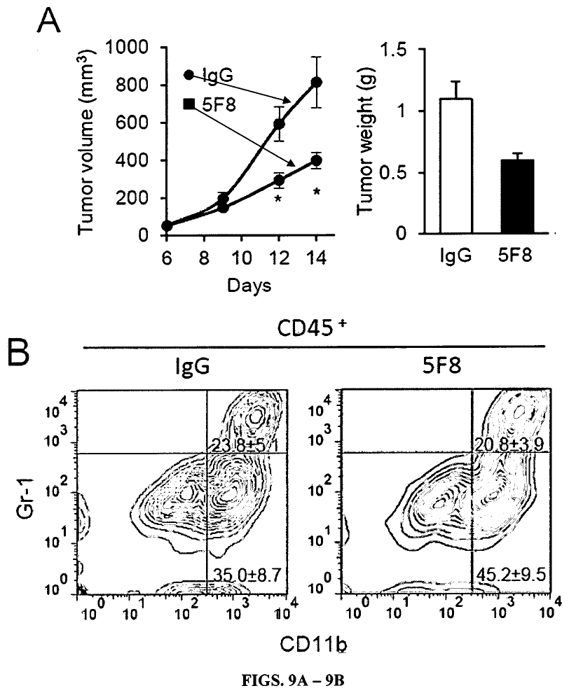

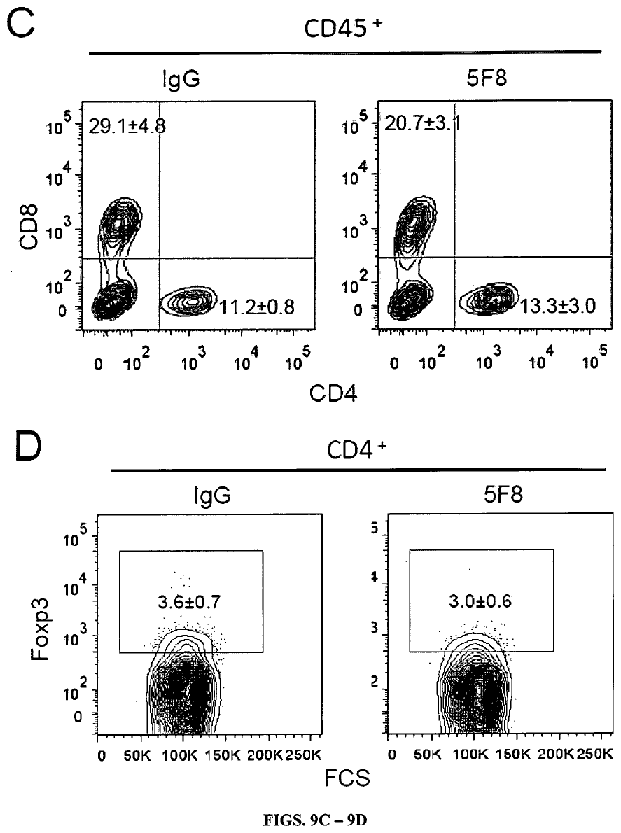

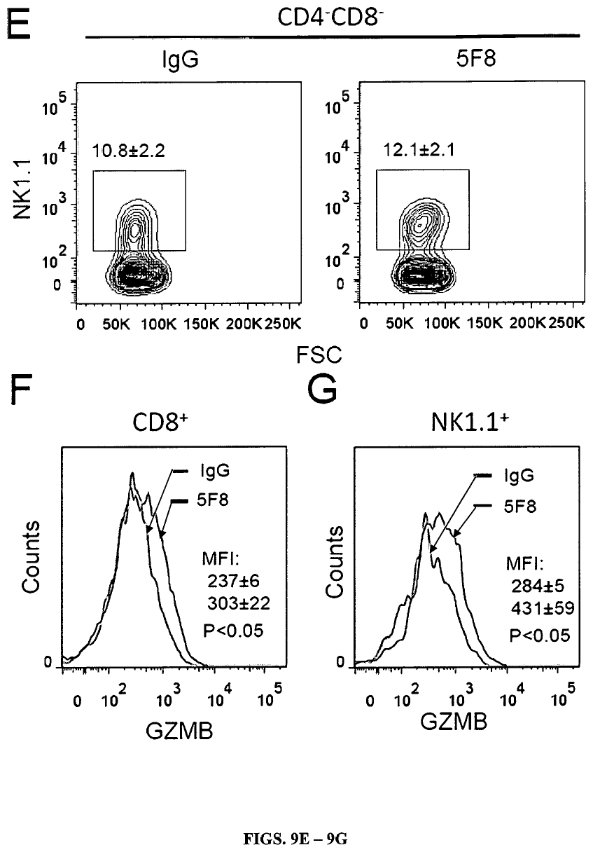

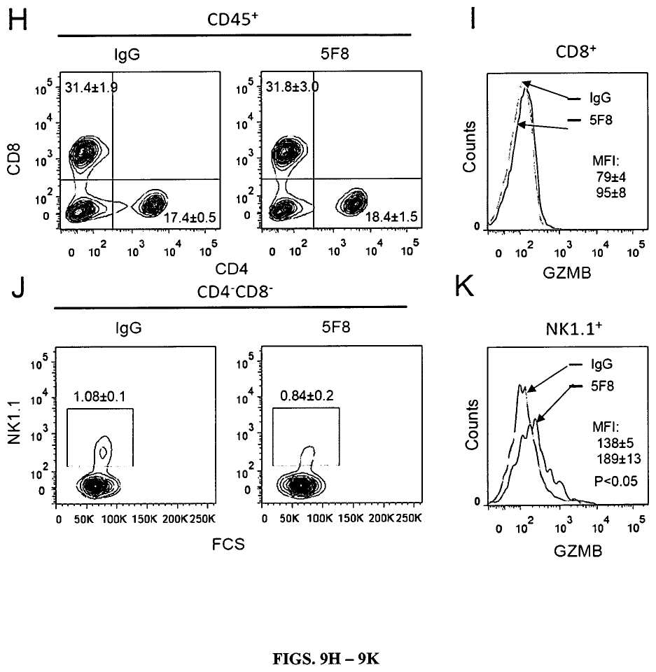

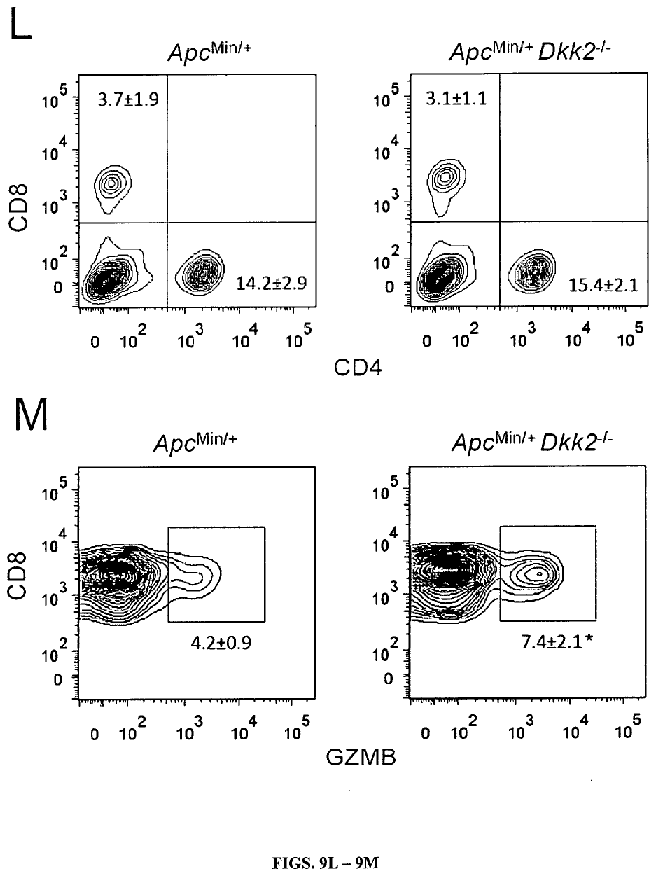

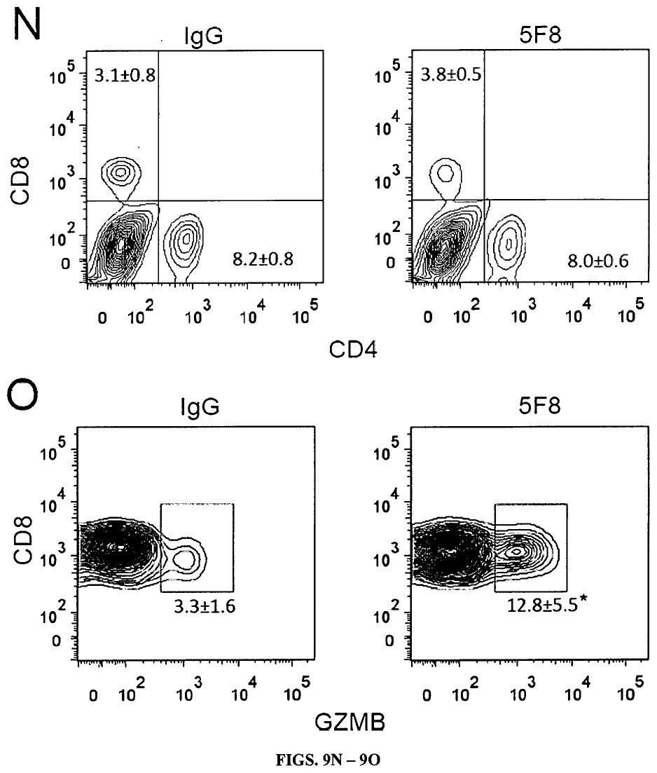

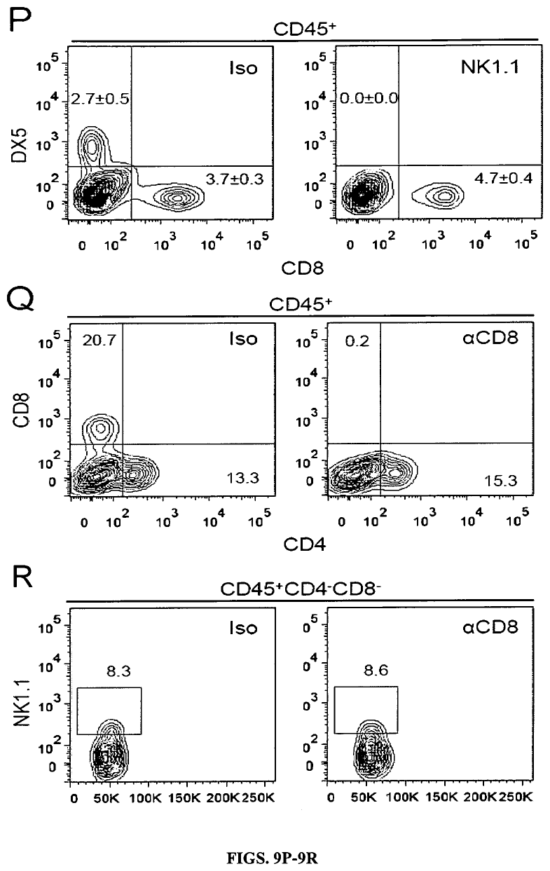

To understand the immune mechanisms, a flow cytometry analysis of tumor-infiltrated leukocytes in antibody-treated MC38 tumors was carried out (FIGS. 9A-9G). There were no significant differences between 5F8 and its isotype-treated samples in the percentage of myeloid cells (Gr1^{high}CD11b^{high} or Gr1^{low}CD11b^{high}), CD4+, CD8+, T regulatory cells (CD4+CD25+Foxp3+), or NK1.1+ cells (FIGS. 9B-9E). However, 5F8 treatment led to significant increases in granzyme B in both CD8+ and NK1.1+ cells (FIGS. 9F-9G). These results are consistent with the immunostaining results (FIG. 2G) and indicate that the granzyme B positive cells detected in the immunostaining are NK and CD8+ T cells. The tumor draining lymph nodes were also analyzed. While there were no significant differences between 5F8 and isotype-treated samples in the populations of CD4+, CD8+, or NK1.1+ cells.

To exclude the effect of tumor size on the flow cytometry results, MC38 tumor-bearing mice were treated with 5F8 and its isotype control for only 24 hours and collected tumor specimens for analysis. At this time point, there was no obvious difference in tumor sizes. While there were still no significant differences in the populations of CD4+, CD8+, or NK1.1+ cells (FIGS. 3C-3D), strong increases in granzyme B were observed in tumor infiltrated CD8+ and NK1.1+ cells in 5F8-treated specimens over isotype-treated ones (FIGS. 3E-3F). Other activation markers of CD8+ and NK cells were examined and significant increases were found in CD69, CD107a, CD314, and CD25, on CD8+ cells and CD69 and CD314 on NK cells (FIGS. 3G-3H). There were also trends of increased IFN_{Y} in CD8+ and NK1.1+ cells (FIGS. 3G-3H) and PD-1 in CD8+ cells (FIG. 3G) in the 5F8-treated specimens. Similar acute 5F8 treatment could also markedly increase granzyme B-positive CD8+ cells in the PPs of the Apc^{Min/+} mice over those treated with the control IgG, without affecting the populations of the T lymphocytes cells (FIGS. 9N-90).

To assess the importance of cytotoxic immune effector cells in DKK2 blockade-mediated tumor suppression, NK cells were depleted with an anti-NK1.1 antibody and CD8+ with an anti-CD8 antibody, respectively, in the MC38 tumor model (FIGS. 9P-9L). Depletion of either NK or CD8+ cells largely diminished the tumor suppressive effect of 5F8 with NK cell depletion perhaps imparting a stronger effect (FIGS. 3I-3J). These results suggest that both NK and CD8+ cells have significant roles in DKK2 blockade-mediated suppression of tumor progression.

### Example 5: DKK2 Directly Suppresses Cytotoxic Immune Cells

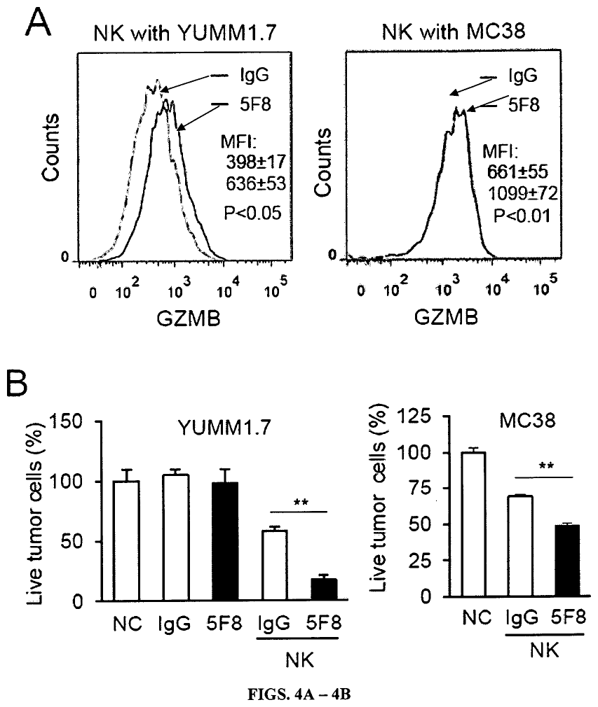

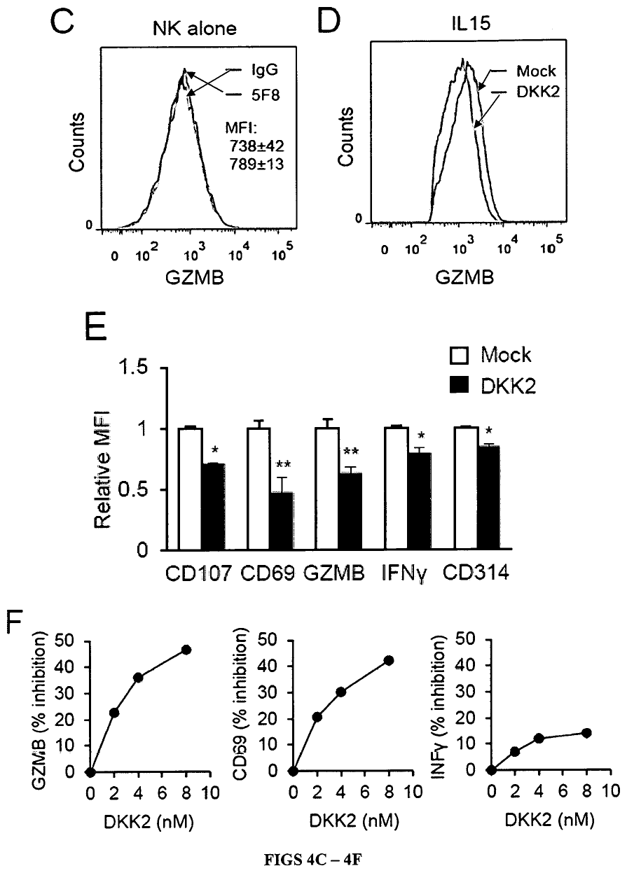

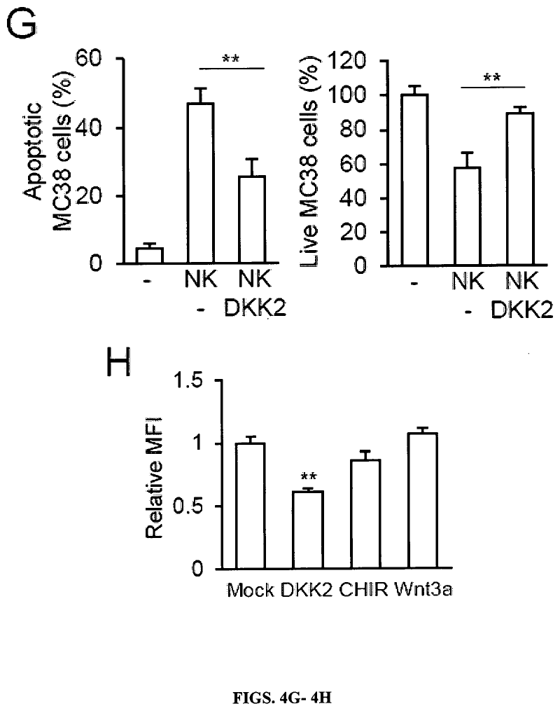

To gain further insights into how the anti-DKK2 antibody suppresses tumor progression, DKK2 blockade was examined for its capacity of promoting tumor cell death in co-culture of tumor cells with primary NK cells. Inclusion of 5F8 caused a marked increase in granzyme B expression in the NK cells (FIG. 4A) and decreases in tumor cell viability (FIG. 4B), when IL-15-expanded primary mouse NK cells were co-cultured with the MC38 cells. By contrast, 5F8 treatment showed little effects on granzyme B expression in the NK cells (FIG. 4C) or the viability of MC38 (FIG. 2C), when these cells were cultured alone.

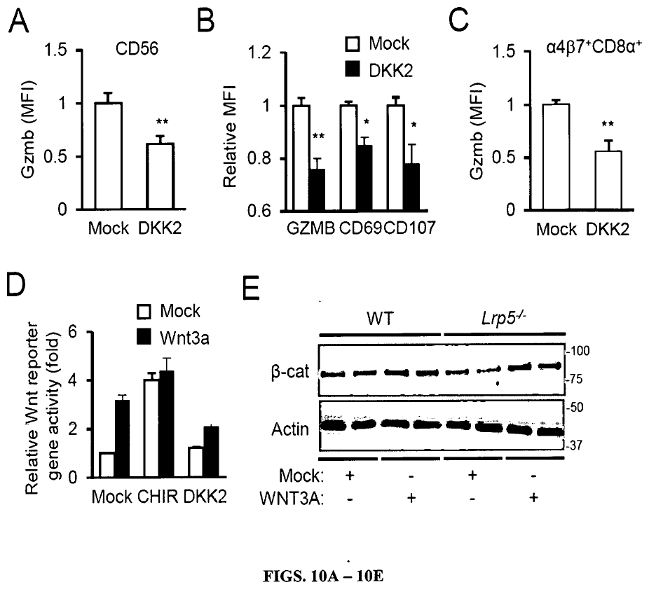

Microarray gene expression analyses were performed and did show significant alteration in the expression of IL-2, IL-15, MHC-I haplotypes, NKG2D ligands (RAE-1a-e, MULT-1, and H60a-c), Fas or TRAILR1/2, all of which are important for NK cell activity, in 5F8-treated MC38 cells or tumors in comparison with isotype IgG-treated ones. In addition, DKK2 mRNA was hardly detectable by RT-PCR in NK cells, whereas DKK2 mRNA was readily detectable in MC38 cells (FIGS. 8D-8E). Together with the aforementioned co-culture results, DKK2, which is produced by tumor cells, may act directly on the NK cells. When recombinant DKK2 protein was added to isolated primary NK cells that were cultured in the presence of IL-15, it caused significant reductions in granzyme B as well as a number of other NK activation markers, including CD69, IFN_{Y}, CD107a, and CD314 (FIGS. 4D-4E). The inhibitory effects of DKK2 on NK cell activation were dose-dependent (FIG. 4F). This effect of DKK2 protein on NK activation markers can be translated into significant impacts on the tumor killing ability, because NK cells pre-treated with DKK2 protein showed reduced ability to cause tumor cell apoptosis and death (FIG. 4G). The DKK2 protein could also inhibit granzyme B expression in human NK cells isolated from peripheral bloods (FIG. 10A). In addition, DKK2 directly suppresses mouse primary CD8+ isolated from spleens (FIG. 10B) and CD8+ intraepithelial cells from intestines (FIG. 10C). Thus, these data together indicate that DKK2 can directly suppress IL-15-mediated NK and CD8+ cell activation.

### Example 6: DKK2 Blockade Enhances NK and CD8⁺ Cell Activation

To understand the immune mechanisms of DKK2 blockade, flow cytometry analysis of tumor infiltrated leukocytes in antibody-treated MC38 tumors was performed (FIG. 9A-G). There were no significant differences between 5F8 and its isotype-treated samples in the percentage of myeloid cells (Gr1^{high}CD11b^{high} or Gr1^{low}CD11b^{high}), CD4⁺, CD8⁺, T regulatory cells (CD4⁺ CD25⁺ Foxp3⁺), or NK1.1⁺ cells (FIG. 9B-E). However, 5F8 treatment led to significant increases in granzyme B in both CD8⁺ and NK1.1⁺ cells (FIG. 9F-G). These results are consistent with the immunostaining results (FIG. 2G) and indicate that the granzyme B positive cells detected in the immunostaining are NK and CD8⁺ T cells. The tumor draining lymph nodes were also analyzed. While there were no significant differences between 5F8 and isotype-treated samples in the populations of CD4⁺, CD8⁺, or NK1.1⁺ cells (FIG. 9H-J), there was a trend towards an increase in granzyme B in CD8⁺ cells (FIG. 9I) and a significant increase in granzyme B in NK1.1⁺ cells in the 5F8-treated samples over control (IgG3) treated samples (FIG. 9K). Peyer's Patches (PPs), which are the draining lymph nodes for intestinal tumors, were also examined for levels of granzyme B. Increases in granzyme B-positive CD8+ T cells were also observed in Peyer's Patches of DKK2^{-/-} APC^{Min/+} mice over those in the APC^{Min/+} mice; however, there was little difference in the population of CD4⁺ or CD8⁺ cells between animals (FIG. 9L-M).

To exclude the effect of tumor size on the flow cytometry results, MC38 tumor-bearing mice were treated acutely with 5F8 or an isotype (IgG) control for only 24 hours. Tumor specimens were collected for analysis. At this time point, there were no obvious differences in tumor sizes between the 5F8 treated and control animals. Consistent with previous data herein, there were no significant differences between the populations of CD4+, CD8+, or NK1.1+ cells (FIG. 3C-D) in treated compared to control; further, there were strong increases in granzyme B in tumor infiltrated CD8+ and NK1.1+ cells in 5F8-treated specimens over isotype-treated ones (FIG. 3E-F). Other activation markers of CD8+ and NK cells were also examined and significant increases in CD69, CD107a, CD314, and CD25, on CD8+ cells and CD69 and CD314 on NK cells were found (FIG. 3G-H). There were also trends of increased IFNγ in CD8+ and NK1.1+ cells (FIG. 3G-H) and PD-1 in CD8+ cells (FIG. 3G) in the 5F8-treated specimens. Similarly, acute 5F8 treatment also markedly increased granzyme B-positive CD8+ cells in the PPs of the APC^{Min/+} mice over those treated with control IgG, without affecting the populations of T lymphocytes cells (FIG. 9N-O).

To assess the importance of cytotoxic immune effector cells in DKK2 blockade-mediated tumor suppression, NK cells were depleted with an anti-NK1.1 antibody and CD8+ with an anti-CD8 antibody, respectively, in the MC38 tumor model. Depletion of either NK or CD8+ cells largely diminished the tumor suppressive effect of 5F8 (FIG. 3I-J); further, the data suggest that NK cell depletion has a stronger effect than CD8+ depletion in counteracting the ameliorative effects of 5F8 on tumor progression (FIG. 3I-J). These results suggest that both NK and CD8+ cells have significant roles in DKK2 blockade-mediated suppression of tumor progression.

### Example 7: DKK2 Inhibits NK Cell Activation Independently of Wnt-β-Catenin Signaling

In view of the fact that DKK2 can inhibit Wnt-β-catenin signaling, the present study tested whether Wnt-β-catenin signaling is responsible for NK cell regulation by DKK2. In a Wnt reporter gene assay, WNT3A protein induced a strong increase in the reporter gene activity, which could be inhibited by DKK2 protein (FIG. 10D). In addition, WNT3A induced β-catenin accumulation in the primary NK cells (FIG. 10E). However, WNT3A had no significant effect on granzyme B expression in the NK cells (FIG. 4H). CHIR99021, a GSK3 inhibitor that increases β-catenin stability bypassing WNT and its receptors, was also tested. Despite its strong effect on Wnt reporter gene activity (FIG. 10D), CHIR99021 showed no significant effect on granzyme B in primary NK cells (FIG. 4H). Therefore, inhibition of DKK2 signaling that results in NK cell activation is not likely due to its effect on Wnt-β-catenin signaling. These results also distinguish the mechanism of action of DKK2 from recent reports indicating an involvement of Wnt-β-catenin signaling in modulation of tumor immune microenvironments (D'Amico, L., et. al. 2016. J. Exp. Med. 213(5):827-40; Malladi, S., et al., 2016. Cell. 165: 45-60).

### Example 8: DKK2 Impedes Phosphorylated STAT5 Nuclear Localization

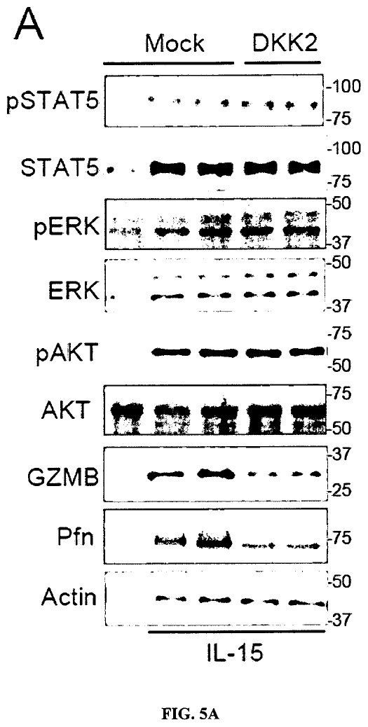

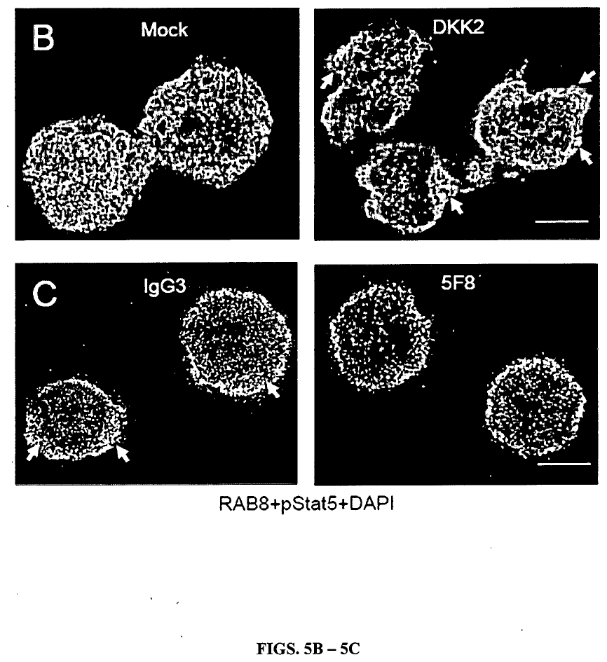

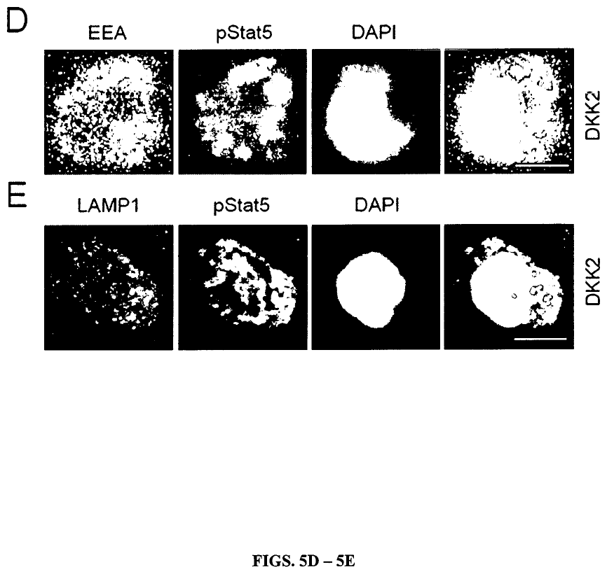

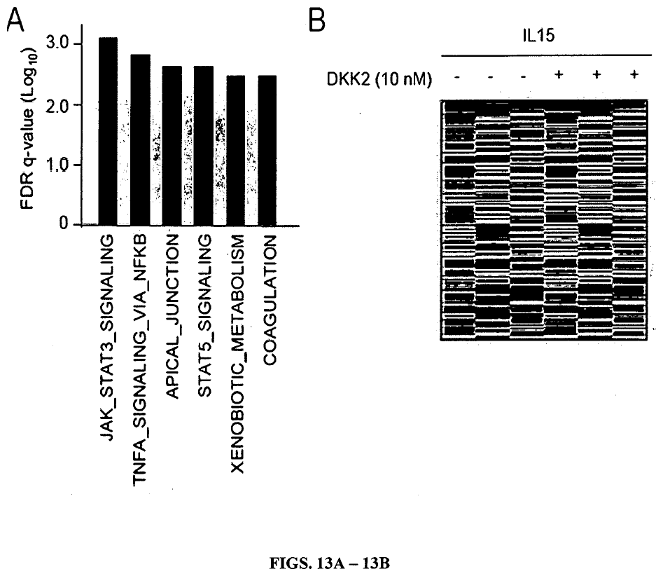

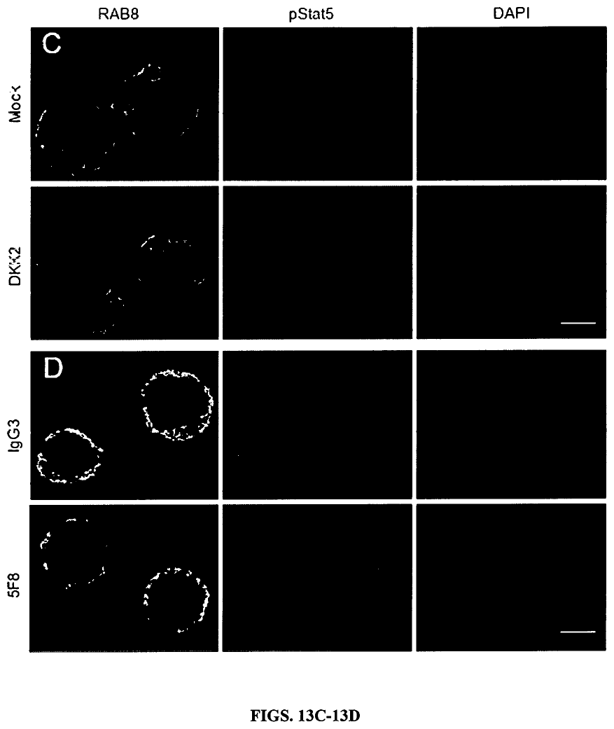

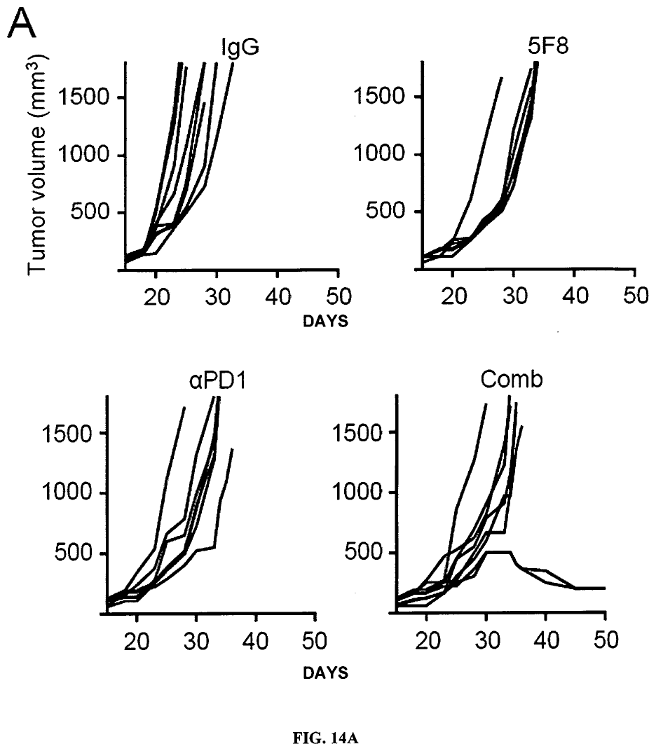

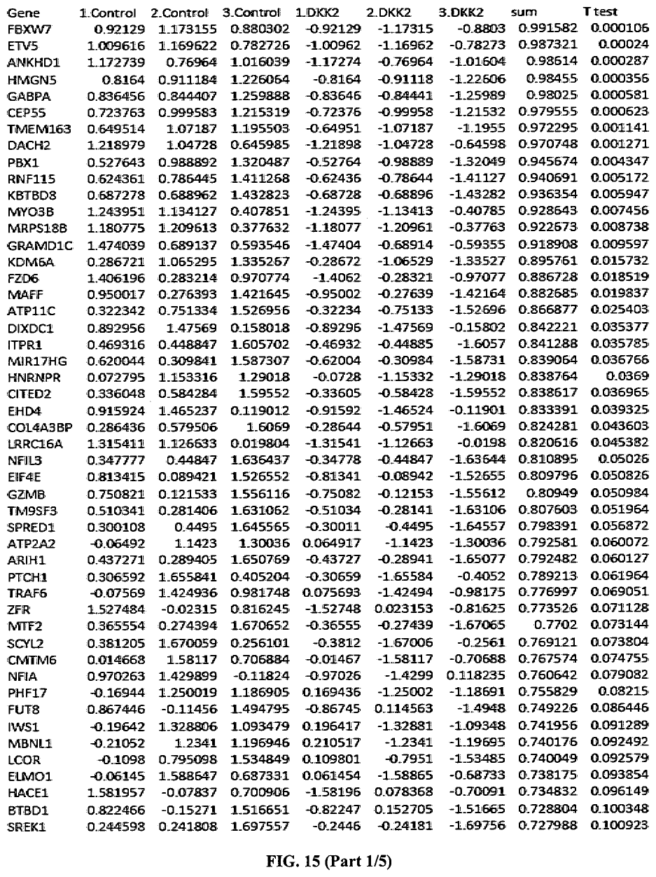

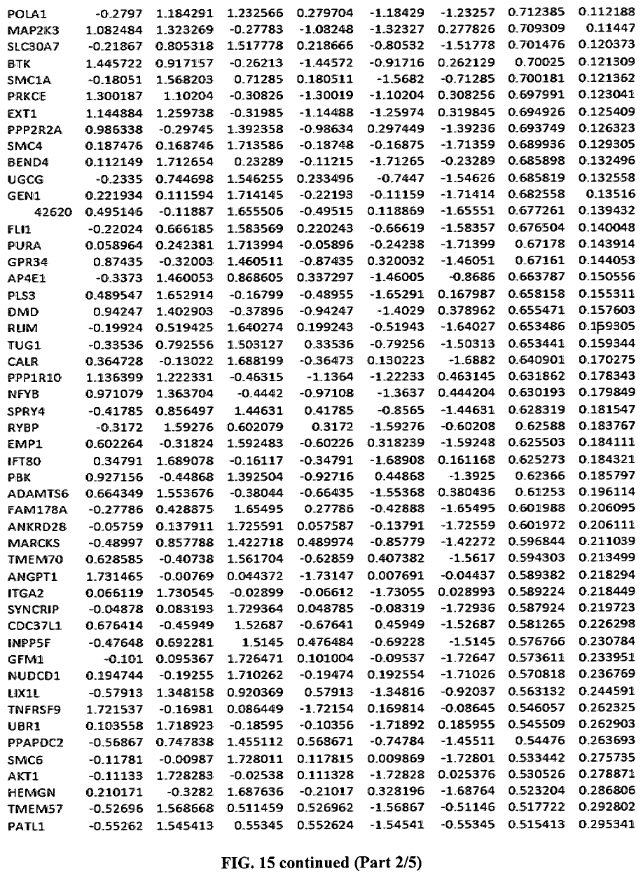

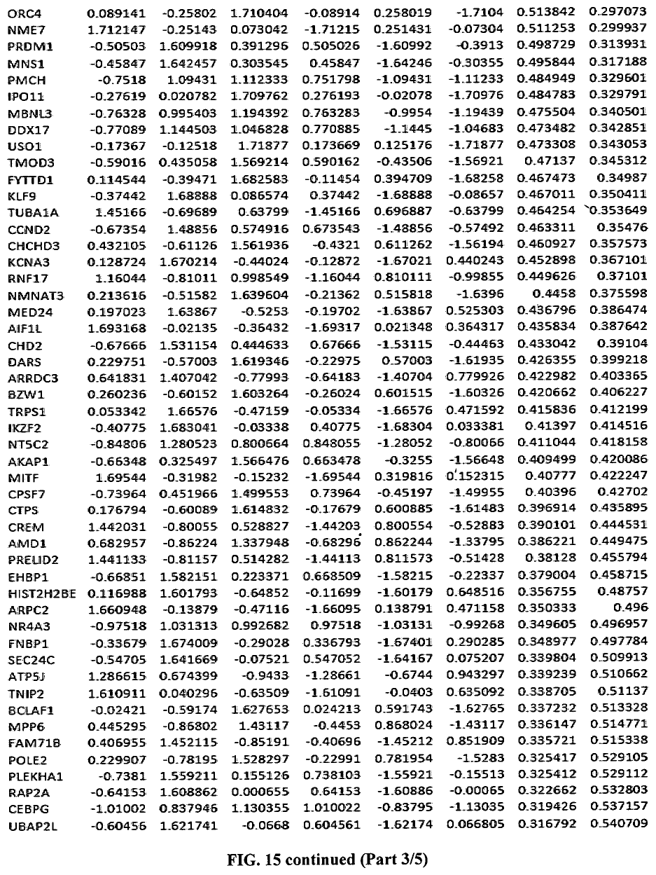

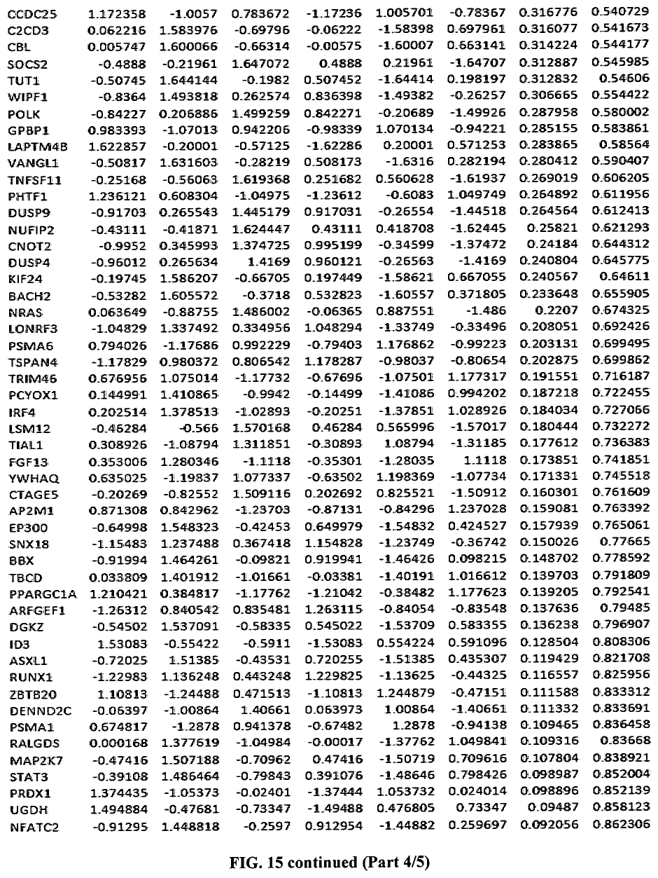

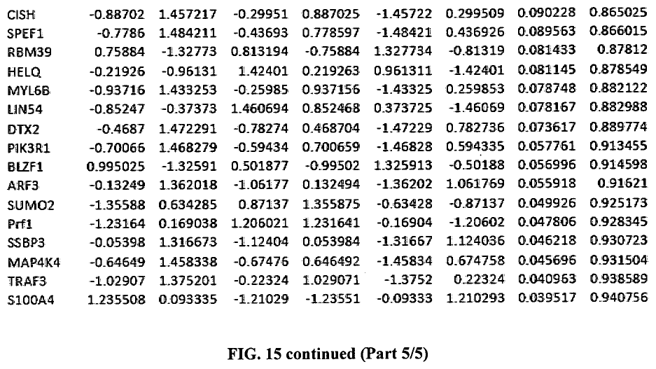

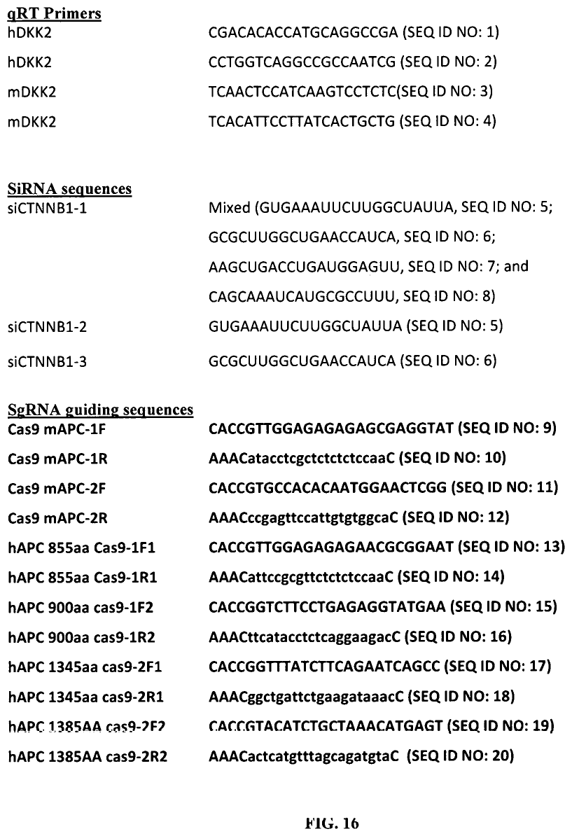

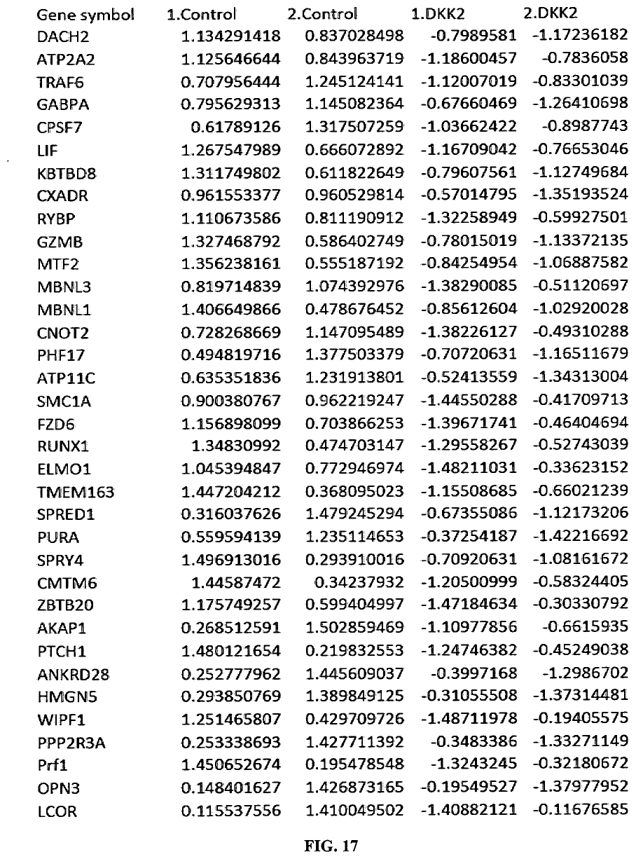

To understand how DKK2 suppresses cytotoxic immune cell activation by IL-15, the effects of DKK2 treatment on various signaling events stimulated by IL-15 were examined. No notable changes in phosphorylated STAT5, ERK, and AKT were detected (FIG. 5A). Consistent with the flow cytometry results, reduced granzyme B was observed in DKK2-treated samples (FIG. 5A). In addition, reduced perforin was observed in DKK2-treated samples (FIG. 5A). However, sequencing of the mRNAs from DKK2-treated primary NK cells in comparison of those from mock-treated cells suggest an alteration in STAT signaling by DKK2 treatment (FIGS. 13A-13B, FIG. 15 and FIG. 17). Next, the localization of phosphorylated-STAT5 (phosphor-STAT) was examined. While IL15 induces nuclear localization of phospho-STAT5 as expected, cytosolic localization of phospho-STAT5 was readily detected in cells treated with DKK2 (FIG. 5B and FIG. 13C). Concordantly, NK cells isolated from 5F8-treated tumors show reduced cytosolic localization of phospho-STAT5 over those isolated from control IgG-treated tumors (FIG. 5C). Concordantly, NK cells isolated from 5F8-treated tumors show reduced cytosolic localization of phospho-STAT5 over those isolated from control IgG-treated tumors (FIG. 5C and FIG. 13D). Phospho-STAT5 appeared to be partially co-localized with early endosome marker, Early Endosome Antigen 1 (EEA1) (FIG. 5D), but not with late endosome marker, Lysosome Associated Membrane Protein 1 (LAMP-1), in DKK2-treated NK cells (FIG. 5E); this data suggests that phospho-STAT5 may be sequestered on early/recycling endosomes, including EEA1-positive early endosomes. Thus, these data indicate that DKK2 treatment does not disrupt the mechanism whereby IL-15 signaling leads to STAT5 phosphorylation, but rather, DKK2 treatment impairs the nuclear localization of phosphorylated STAT5.

### Example 9: DKK2 Acts Through LRP5, but not LRP6

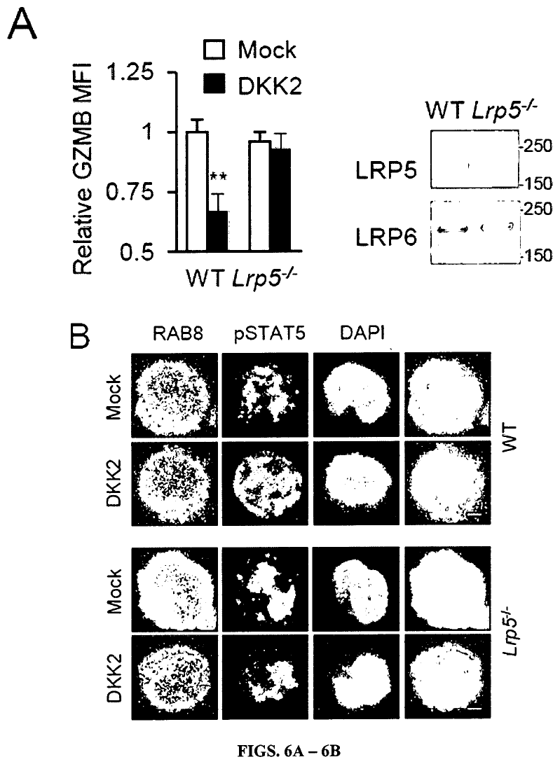

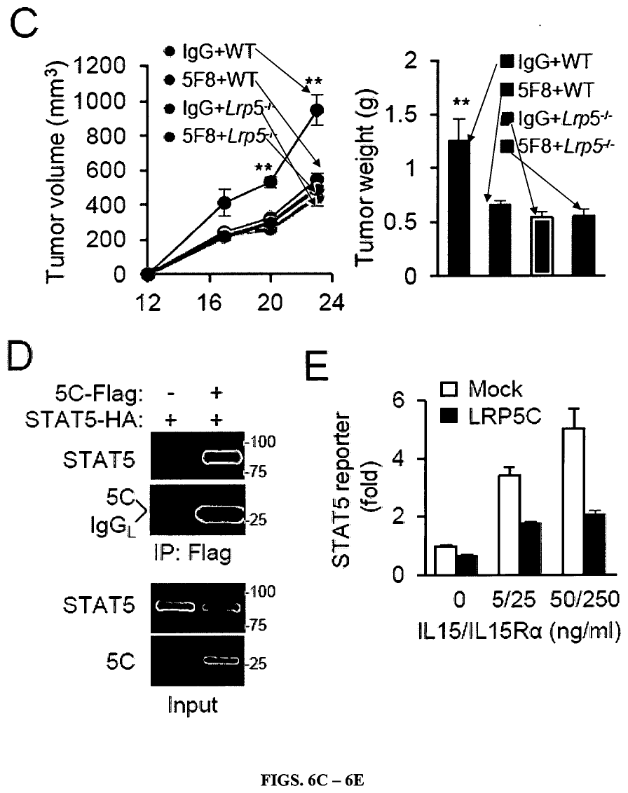

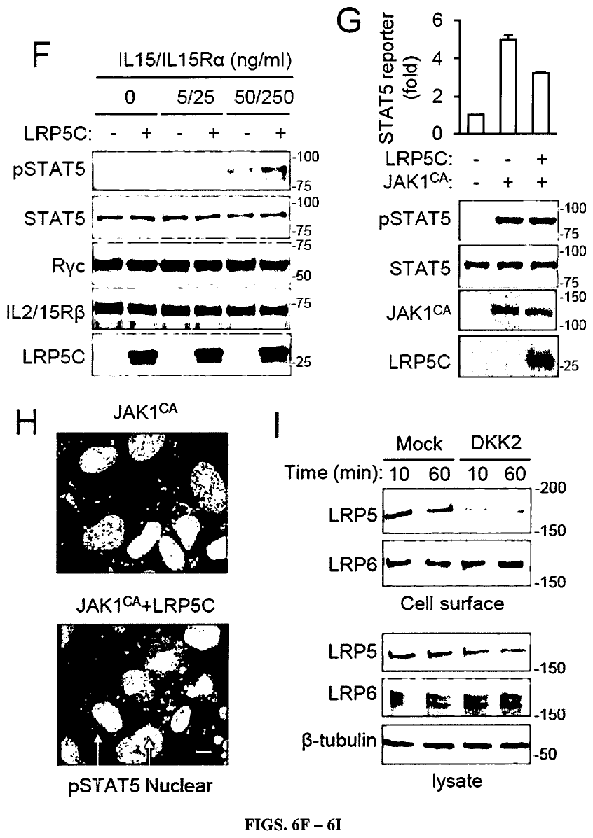

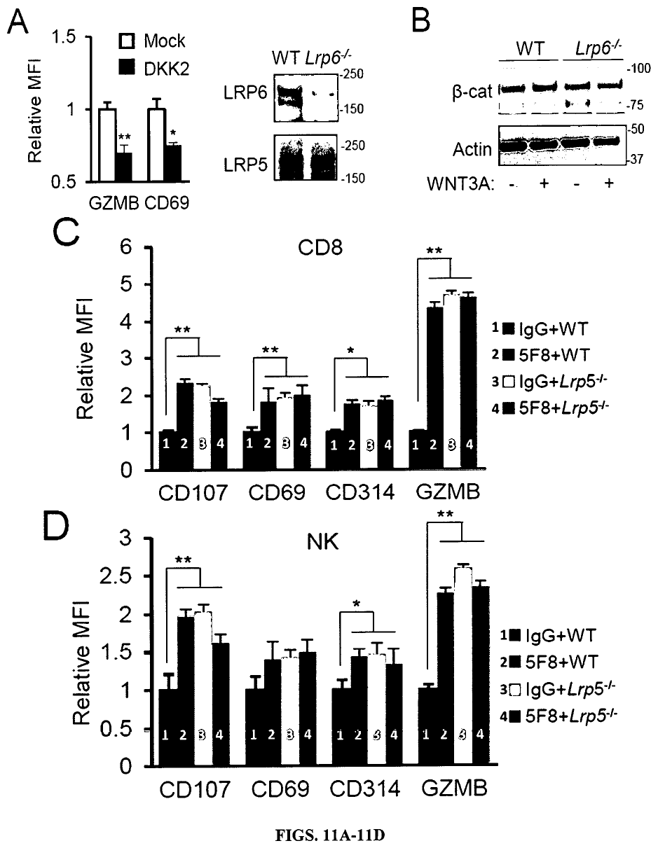

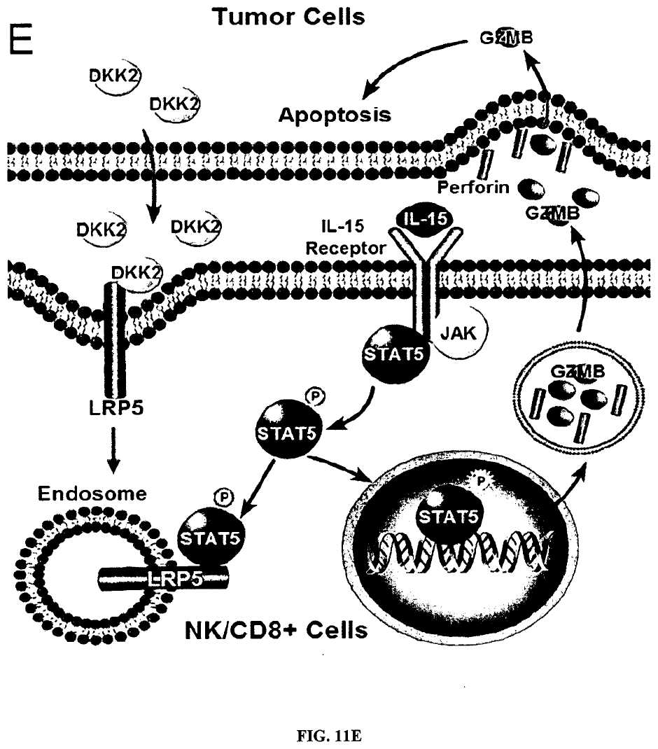

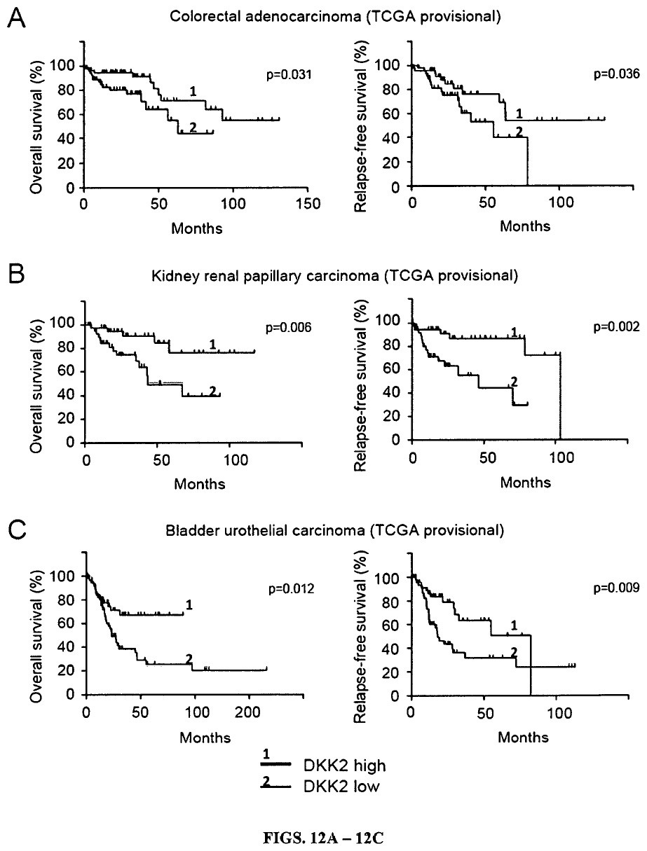

DKK2 binds to LRP5 and LRP6. While DKK2 could still inhibit the activation of primary NK cells lacking LRP6 (FIG. 11A), it failed to inhibit LRP5-deficient NK cells (FIG. 6A). Additionally, DKK2 failed to cause impairment of phospho-STAT5 nuclear localization in NK cells lacking LRP5 (FIG. 6B). Taken together, these results indicate that LRP5, but not LRP6, is required for DKK2's action on NK cells. The fact that LRP5-deficiency did not affect β-catenin accumulation stimulated by Wnt3A in the NK cells (FIG. 10E), further confirms that the effect of the DKK2-LRP5 axis on NK activation is independent of Wnt-β-catenin signaling. In contrast, LRP6 plays a key role in Wnt-β-catenin signaling in NK cells, as WNT3A did not induce β-catenin accumulation in LRP6-deficient NK cells (FIG. 11B).

To further test the importance of LRP5 in tumor progression and the anti-tumor effect of DKK2 blockade, an adoptive cell transfer model was employed. Specifically, bone marrows (BMs) from the Lrp5fl/flMx1Cre mice were transferred into lethally irradiated WT C57BL mice. After recovery and Cre expression induction, the mice were grafted with the MC38 cells. The lack of LRP5 in hematopoietic cells led to a significant impediment in the progression of the grafted tumors (FIG. 6C). Importantly, the anti-DKK2 antibody 5F8 showed no significant effects on the tumor progression, while it still retained its tumor suppressive effect in mice received WT BM transfer (FIG. 6C). Flow cytometry analysis of tumor infiltrated leukocytes provides results consistent with the conclusion that 5F8 exerts its effects on cytotoxic immune cell activation and tumor suppression via LRP5; further, LRP5 deficiency in hematopoietic cells phenocopies 5F8 treatment in cytotoxic immune cell activation and abrogated 5F8's effects on cytotoxic immune cell activation (FIGS. 11B-11C). These data, together with the data in FIG. 1F, establish that an LRP5 specific antibody that would optimally suppress tumor formation is useful because it would both: (i) block DKK2 binding to/signaling via LRP5 and (ii) have no effect on Wnt signaling, which is primarily mediated by the Wnt co-receptor LRP6.

### Example 10: LRP5C Interacts with and Inhibits STAT5

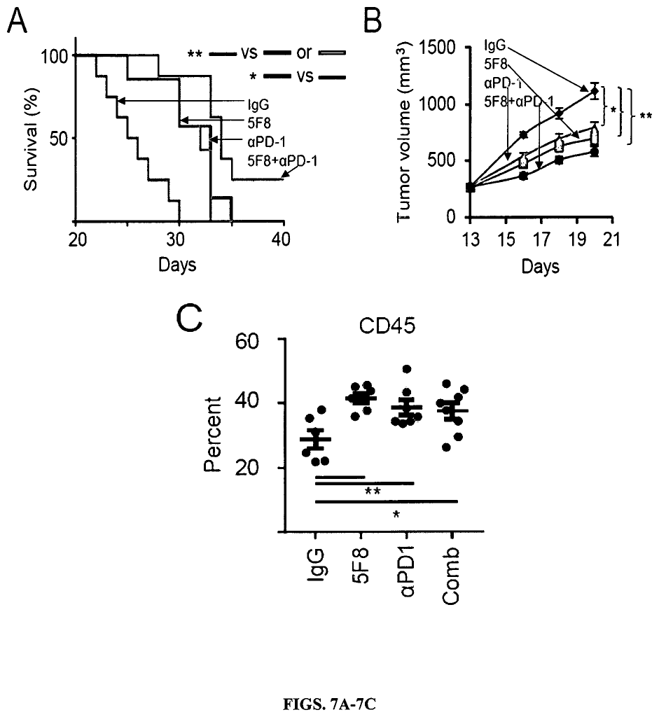

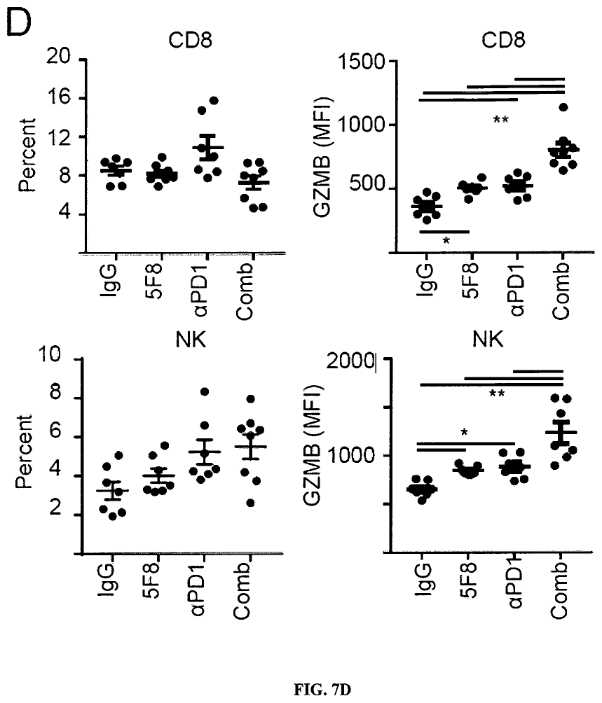

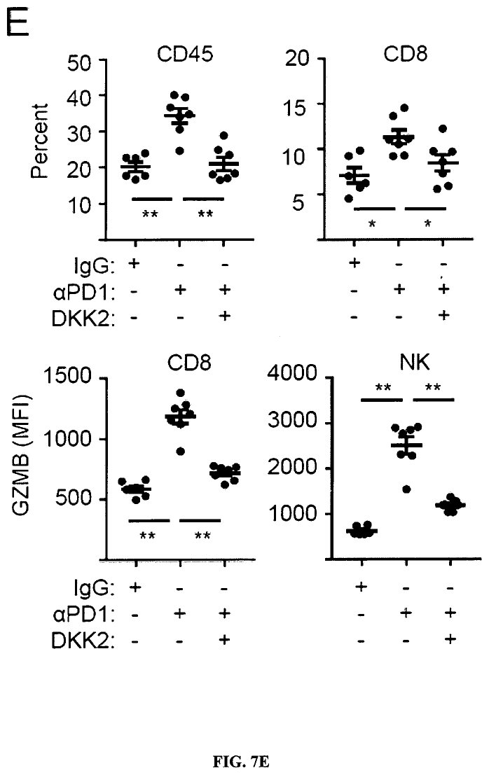

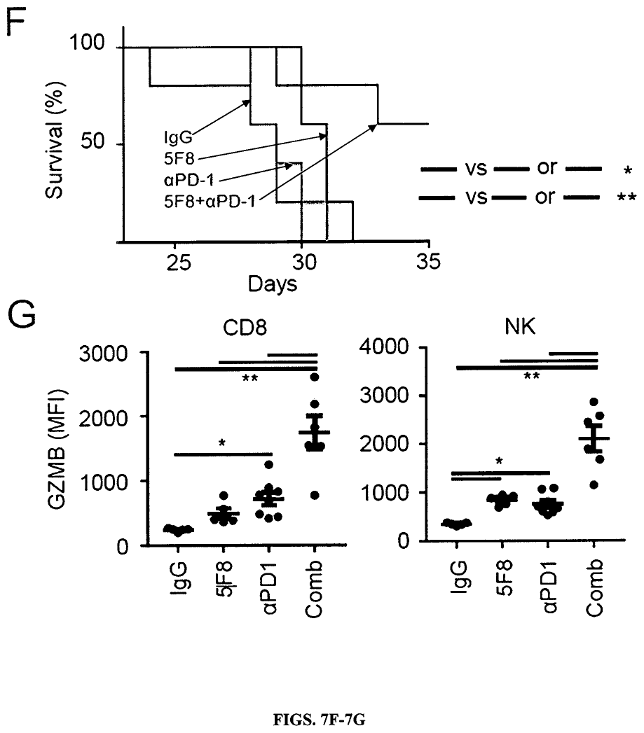

To better understand how LRP5 interferes with phosphorylated STAT5 nuclear localization, the interaction between LRP5 intracellular domain (LRP5C) and STAT5 was examined. LRP5C and STAT5 co-immunoprecipitated in HEK293 cells (FIG. 6D). Next, the effect of LRP5C on IL-15-mediated activation of STAT5 reporter gene activity was tested in HEK293 cells expressing JAK3, 1L2/15 β and common γ receptor subunits in these cells. Expression of LRP5C markedly inhibited the STAT5 reporter gene activity (FIG. 6E) without affecting STAT5 phosphorylation (FIG. 6F) when stimulated with IL-15. Furthermore, LRP5C could inhibit the STAT5 reporter gene activity stimulated by the expression of a constitutively active JAK1 mutant (V658F) (Haan, C. et al., 2011. Chem. Biol. 18: 314-323) in HEK293 cells (FIG. 6G). While LRP5C expression did not alter STAT5 phosphorylation (FIG. 6G), it impaired the nuclear localization of phosphorylated STAT5 induced by activated JAK1 expression (FIG. 6H). These data are consistent with observations made in the primary NK cells and support the conclusion that DKK2 inhibits IL-15 signaling by impeding the nuclear localization of phosphorylated STAT5 via LRP5C interaction with STAT5. The observation that DKK2 induces rapid internalization of LRP5 rather than LRP6 (FIG. 6I) provides additional support to the mechanism depicted in FIG. 7 by which DKK2 induces phosphorylated STAT5 cytosolic sequestration at endosomes through internalized LRP5, but not LRP6.

### Example 11: DKK2 Suppresses Tumor Immune Response to Anti-PD-1

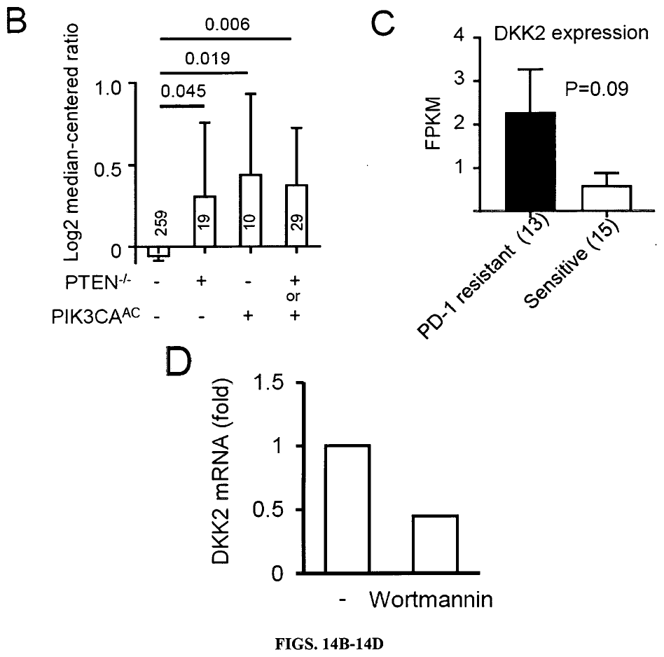

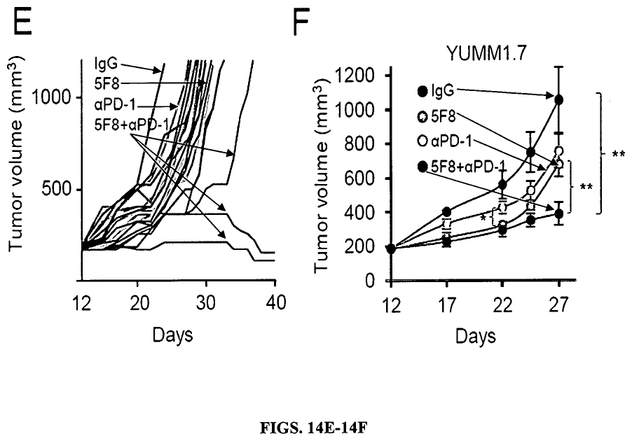

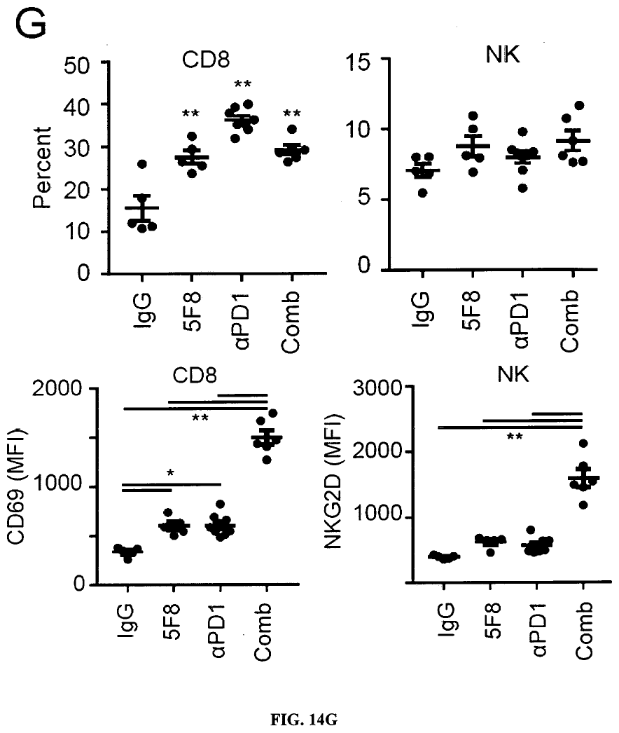

To evaluate the therapeutic potential of DKK2 blockade, the effect of the combination of the DKK2 blockade with the PD-1 blockade was tested using the MC38 tumor model. While both PD-1 and DKK2 blockade showed tumor suppressive effects, the combination yielded further anti-tumor effects (FIGS. 7A-7B and FIG. 14A). Notably, a small fraction of tumors treated with the combination showed complete regression (FIG. 14A). Flow analysis showed that while individual blockades led to increases in granzyme B levels in tumor infiltrated CD8+ and NK cells, the combination blockade resulted in further increases in granzyme B levels in these cells (FIGS. 7B-7D). To more directly assess the effect of DKK2 on tumor immune responses elicited by PD-1 blockade, intratumoral administration of recombinant DKK2 protein was performed. The DKK2 protein inhibited PD-1 blockade-induced increases in the numbers of tumor-infiltrated CD45+ and CD8+ cells and activation of CD8+ and NK cells (FIG. 7E). These results together provides an explanation for the additional tumor suppressive effects of the combination blockade.

Analysis of the skin cutaneous melanoma (TCGA, Provisional) cohort revealed correlations of PTEN-loss of function and PI3K gain of function mutations with elevated DKK2 expression (FIG. 14B). These mutations leads to increases in the cellular levels of phosphatidylinositol (3,4,5)-trisphosphate. In addition, a trend of correlation of PD-1 resistance with increased DKK2 expression (FIG. 14C) (Hugo et al., Cell 165, 35-44 (2016)) and significant correlation with PTEN-loss mutations (Peng et al., Cancer Discov 6, 202-216 (2016)) were observed in human melanomas. Thus tested the anti-tumor effect of DKK2 blockade was tested in comparison of PD-1 blockade or their combination using the YUMM1.7 mouse melanoma cells. The YUMM1.7 cells were derived from Braf^{V600E}Pten^{-/-}Cdkn2a^{-/-} melanoma developed in the C57BL/6 mice (Kaur et al., Nature, (2016)). The level of DKK2 mRNA in YUMM1.7 cells, which is more than 10 folds higher than that in MC38 cells, could be reduced by PI3K inhibitor Wortmannin treatment (FIG. 14D), suggesting that DKK2 expression is regulated by phosphatidylinositol (3,4,5)-trisphosphate elevation. Importantly, the anti-DKK2 antibody significantly impeded tumor progression and extended the survival of tumor-bearing mice in the YUMM1.7 tumor model (FIG. 7F and FIGS. 14E-14F). In addition, DKK2 blockade showed a general trend of better performance than PD-1 blockade (FIG. 7F and FIGS. 14E-14F). While the combination showed a stronger survival benefit than the individual blockade when they are compared to the control in the MC38 model, it significantly outperformed the individual blockade for the YUMM1.7 melanoma model (FIG. 7F and FIGS. 14E-14F). In addition, a fraction of mice treated with the combination showed complete regression (FIG. 14E). Flow analysis of tumor infiltrated leukocytes show significant stronger activation of CD8+ and NK cells by the combination of DKK2 and PD-1 blockades (FIG. 7G and FIG. 14G). These data support the conclusion alluded earlier and suggest a broader applicability of DKK2 blockade in tumor therapies.

### Example 12: Summary

In this study, previously unknown function of DKK2 in promoting tumor progression was uncovered. Its blockade was shown to lead to suppression of tumor progression in mouse models. DKK2 blockade-mediated tumor suppression was shown to depend upon the host immune system, in particular NK and CD8+ cells. DKK2 was shown to have the capacity of directly inhibiting NK and CD8+ cell activation by IL-15 and characterized a mechanism for this action of DKK2. In this mechanism, DKK2 impedes nuclear localization of phosphorylated STAT5 specifically through LRP5, but not LRP6 (FIG. 11E). DKK2 can bind to LRP5 (FIG. 1F) and LRP6 (Li et al., PNAS 109, 1140211407 (2012)), which are both expressed in NK cells. It remains unclear why only LRP5 is required for the DKK2 actions here. Knowing that DKK2 induces sequestration of phospho-STAT5 at endosomes, the ability of LRP5 to be internalized in response to DKK2 in contrast of LRP6 (FIG. 6I) may provide an explanation. The failure of LRP6 to be internalized upon ligand binding has been previously reported; both DKK1 (Semenov, et al., J Biol Chem, (2008)) and WNT3A (Kim et al., J Cell Biol. 200, 419-428 (2013)) was shown not to induce internalization of endogenous LRP6. One key difference between LRP5 and LRP6 pertinent to their ability for internalization is that LRP5 has three putative adaptor protein-2 (AP2)-binding motifs in contrast to one such motif in LRP6 in their intracellular domains as noted previously (Kim et al., J Cell Biol. 200, 419-428 (2013)). AP2 is a component in clathrin-mediated endocytosis, and one of its functions is cargo recognition (McMahon et al., Nat Rev Mol Cell Biol 12, 517-533 (2011), Ohno, J Cell Sci 119, 3719-3721 (2006)).

DKK2 may exert more potent effects on NK cells in tumors than in the in vitro assays because the LRP5 mRNA level is eight times higher in tumor infiltrated NK cells than primary NK cells isolated form spleens as determined by quantitative RT-PCR. Nevertheless, DKK2 protein exert a clear effect on phosphorylated STAT5 nuclear localization in vitro. However, DKK2 does not appear to cause complete exclusion of phosphorylated STAT5 from the nuclei (FIG. 5B). This partial effect on phosphorylated STAT5 nuclear localization may explain why DKK2 only has a partial, but biologically significant, effect on the activation of NK cells, while lacking a strong effect on NK cell development. This may also explain why DKK2 inhibition does not alter NK cell number in mice, given that deficiency of STAT5 or IL-15 signaling-specific IL15 receptor a subunit has a profound effect on NK cell development (K. Imada et al., J Exp Med 188, 2067-2074 (1998); S. Teglund et al., Cell 93, 841-850 (1998); E. Eckelhart et al., Blood 117, 1565-1573 (2011)). These results may also be interpreted to suggest that NK cell development and full activation have different thresholds for STAT5 signaling. Consistent with this idea, DKK2 seems to show different degree of inhibition of interferon gamma than granzyme B in IL-15 activated NK cells (FIG. 4F). DKK2 can also inhibit IL-15-mediated activation of CD8+ cells (FIG. 10B) presumably through a mechanism similar to its inhibition of STAT5 signaling in the NK cells. Of note, LRP5 expression was reported to be upregulated in human mature CD8+ cells (Wu et al., Immunity 26, 227-239 (2007)), suggesting that DKK2 may also have a stronger effect on CD8+ cells in vivo. However, DKK2 does not inhibit T cell receptor-mediated activation of the primary T cells. This provides an explanation for the lack of an effect of DKK2 blockade on T cell populations. It also suggests that the activation of CD8+ cells observed with DKK2 blockade in mice could be due to a combination of DKK2's direct and NK cell-mediated indirect regulation of CD8+ cells. While IL-15-STAT5 signaling has a direct role in activation of cytotoxic CD8+ T cells, NK cells can also enhance adapted anti-tumor immunity (Crouse et al., Trends Immunol 36, 49-58 (2015)). Consistent with the prominent role of IL-15-STAT5 signaling in CD8+ intraepithelial cells (Mishra et al., Clin Cancer Res 20, 2044-2050 (2014)), DKK2 was able to inhibit CD8+ IELs isolated from mouse intestines. The direct inhibition of IELs by DKK2 may have a larger role in DKK2 blockade-associated increases in granzyme B-positive CD8+ cells in the Apc^{Min/+} intestinal tumor model. In summary, the potent anti-tumor effect of DKK2 in vivo may be the results of not only its direct effects on NK and CD8+ cells, but also the interaction of these immune cells, and the relative contributions of these mechanisms may be context-dependent.

Search of the Gene Expression Atlas (www.ebi.ac.uk/gxa/home) reveals that DKK2 is generally expressed at low levels in various normal human and mouse tissues particularly immune tissues. This suggests that DKK2 inhibition may not be a strong risk factor for increasing autoimmunity. Indeed, DKK2-deficiency does not alter various hematopoietic cell populations in mice housed under a specific pathogen-free condition up to 12 months. As demonstrated in this study, DKK2 expression can be driven by the loss of APC in both human and mouse colon cells (FIGS. 8A-8G). Because direct administration of DKK2 protein into tumors could suppress immune responses elicited by PD-1 blockade (FIG. 7E), the presence of DKK2 in tumors, including the APC-null tumors, would constitute a mechanism for resistance to PD-1 blockade. One explanation for DKK2 to impede the effect of PD-1 blockade may be due to the need of both STAT5 signaling, which is affected by DKK2 blockade, and TCR signaling, which is affected by the PD-1 blockade, but not DKK2 blockade, for full activation antitumor immunity. This may also explain the additional anti-tumor effect of DKK2 and PD-1 blockade combination and be a reason for poor efficacy of PD-1 blockade in human CRC treatment.

DKK2 expression is also be regulated by mechanisms other than APC loss. In human melanomas, DKK2 expression is correlated with mutations leading to phosphatidylinositol (3,4,5)-trisphosphate elevation (FIG. 14B). There is also a trend of upregulation of DKK2 expression in PD-1-resistant human melanomas (FIG. 14C). In addition, significant correction of PTEN-loss with resistance of PD-1 therapy has recently been reported in human melanomas (Peng et al., Cancer Discov 6, 202-216 (2016)). The relationship between PTEN-loss and DKK2 expression was also observed in a mouse melanoma cell line (YUMM1.7) that was derived from a genetically engineered melanoma model harboring a PTEN loss mutation (FIG. 14D). The strong anti-tumor effects of DKK2 blockade and particularly the combination of DKK2 and PD-1 blockade in the YUMM1.7 tumor model suggest that DKK2 blockade may be used to treat PD-1 resistant melanomas and/or to enhance the efficacy of PD-1 blockade in treatment of melanomas harboring phosphatidylinositol (3,4,5)-trisphosphate elevation mutations. Analysis of the TCGA provisional database also revealed significant correlations of high DKK2 expression with poor survival rates for renal papillary carcinoma and bladder urothelial carcinoma (FIG. 14H). DKK2 blockade may thus also be applied for the treatment of these human cancers as single therapy or in combination with other checkpoint inhibitors. These possibilities and the potential of blockade of DKK2 receptor LRP5 in human cancer therapy warrant further investigation in future.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a cancer in a subject in need thereof, the method comprising administering to the subject an effective amount of an inhibiting agent that blocks the interaction between Dickkopf 2 (DKK2) and Low-Density Lipoprotein (LDL) Receptor Related Protein 5 (LRP5), in a pharmaceutical acceptable carrier.

2. A method for providing anti-tumor immunity in a subject, the method comprising administering to the subject an effective amount of an inhibiting agent that blocks the interaction between Dickkopf 2 (DKK2) and Low-Density Lipoprotein (LDL) Receptor Related Protein 5 (LRP5), with a pharmaceutical acceptable carrier.

3. A method for stimulating a T cell-mediated immune response to a cell population or tissue in a subject, the method comprising administering to the subject an effective amount of an inhibiting agent that blocks the interaction between Dickkopf 2 (DKK2) and Low-Density Lipoprotein (LDL) Receptor Related Protein 5 (LRP5), with a pharmaceutical acceptable carrier.

4. A method for stimulating a Natural Killer (NK) cell immune response to a cell population or tissue in a subject, the method comprising administering to the subject an effective amount of an inhibiting agent that blocks the interaction between Dickkopf 2 (DKK2) and Low-Density Lipoprotein (LDL) Receptor Related Protein 5 (LRP5), with a pharmaceutical acceptable carrier.

5. The method of claim 1, wherein the inhibiting agent is at least one selected from the group consisting of a DKK2 antagonist or fragment thereof, a DKK2 antibody or fragment thereof, a LRP5 antagonist or fragment thereof, a LRP5 antibody or fragment thereof, a siRNA, a ribozyme, an antisense molecule, an aptamer, a peptidomimetic, a small molecule, a CRISPR/Cas9 editing system and a combination thereof.

6. The method of claim 1, wherein the inhibiting agent is DKK2 antibody 5F8.

7. A method of treating a cancer in a subject in need thereof the method comprising administering to the subject an effective amount of a Low-Density Lipoprotein (LDL) Receptor Related Protein 5 (LRP5) gene depleting agent in a pharmaceutical acceptable carrier.

8. The method of claim 7, wherein the LRP5 depleting agent is selected from the group consisting of a LRP5 antibody, a siRNA, a ribozyme, an antisense molecule, an aptamer, a peptidomimetic, a small molecule, a CRISPR/Cas9 editing system and a combination thereof.

9. The method of claim 7, wherein the LRP5 depleting agent possesses neutralizing activity.

10. The method of claim 1, wherein the LRP5 depleting agent does not affect canonical Wnt/β-catenin signaling.

11. The method of claim 8, wherein the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

12. The method of claim 7, wherein the cancer is selected from the group consisting of colorectal cancer, pancreatic cancer, gastric cancer, intestinal cancer, pancreatic cancer, esophageal cancer, skin cancer and lung cancer.

13. The method of claim 7, further comprising administering to the subject an additional agent selected from the group consisting of a chemotherapeutic agent, an anti-cell proliferation agent, an immunotherapeutic agent and any combination thereof.

14. The method of claim 13, wherein the additional agent is a programmed cell death 1 (PD-1) antibody.

15. The method of claim 13, wherein the LRP5 depleting agent and the additional agent are co-administered to the subject.

16. The method of claim 7, wherein the route of administration is selected from the group consisting of inhalation, oral, rectal, vaginal, parenteral, topical, transdermal, pulmonary, intranasal, buccal, ophthalmic, intrathecal, and any combination thereof.

17. A pharmaceutical composition for treating a cancer in a subject the pharmaceutical composition comprising a LRP5 depleting agent and a pharmaceutical acceptable carrier.

18. The pharmaceutical composition of claim 17, wherein the LRP5 depleting agent possesses neutralizing activity.

19. The pharmaceutical composition of claim 17, wherein the LRP5 depleting agent does not affect canonical Wnt/β-catenin signaling.

20. The pharmaceutical composition of claim 17, wherein the LRP5 depleting agent is selected from the group consisting of a LRP5 antibody, a siRNA, a ribozyme, an antisense molecule, an aptamer, a peptidomimetic, a small molecule, a CRISPR/Cas9 editing system and a combination thereof.

21. The pharmaceutical composition of claim 20, wherein the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

22. The pharmaceutical composition of claim 11, comprising an additional agent selected from the group consisting of a chemotherapeutic agent, an anti-cell proliferation agent, an immunotherapeutic agent and any combination thereof.

23. The pharmaceutical composition of claim 22, wherein the additional agent is a programmed cell death 1 (PD-1) antibody.

24. The pharmaceutical composition of claim 22, wherein the cancer is selected from the group consisting of colorectal cancer, pancreatic cancer, gastric cancer, intestinal cancer, pancreatic cancer, esophageal cancer, skin cancer and lung cancer.

25. A method for providing anti-tumor immunity in a subject, the method comprising administering to the subject an effective amount of a LRP5 antibody or fragment thereof with a pharmaceutical acceptable carrier.

26. The method of claim 25, wherein the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

27. The method of claim 25, further comprising further administering to the subject an additional agent selected from the group consisting of a chemotherapeutic agent, an anti-cell proliferation agent, an immunotherapeutic agent and any combination thereof.

28. The method of claim 27, wherein the additional agent is a programmed cell death 1 (PD-1) antibody.

29. The method of claim 27, wherein the LRP5 antibody and the additional agent are co-administered to the subject.

30. A method for stimulating a T cell-mediated immune response to a cell population or tissue in a subject, the method comprising administering to the subject an effective amount of a LRP5 antibody or fragment thereof with a pharmaceutical acceptable carrier.

31. The method of claim 30, wherein the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

32. The method of claim 30, wherein the T cell-mediated immune response is a CD8⁺ cytotoxic T lymphocyte (CTL) response.

33. A method for stimulating a Natural Killer (NK) cell immune response to a cell population or tissue in a subject, the method comprising administering to the subject an effective amount of a LRP5 antibody or fragment thereof with a pharmaceutical acceptable carrier.

34. The method of claim 33, wherein the LRP5 antibody comprises an antibody selected from the group comprising a polyclonal antibody, monoclonal antibody, humanized antibody, synthetic antibody, heavy chain antibody, human antibody, biologically active fragment of an antibody, an antibody mimic and any combination thereof.

35. The method of claim 1, wherein the subject is a mammal.

36. The method of claim 35, wherein the mammal is a human.
